(19) United States
(12) Patent Application Publication
(10) Pub. No.: US 2024/0116941 A1
(43) Pub. Date: Apr. 11, 2024

(54) SOLID FORMS OF A HBV CORE PROTEIN ALLOSTERIC MODIFIER

(71) Applicant: Hoffmann-La Roche Inc., Little Falls, NJ (US)

(72) Inventors: Jing Xiong, Shanghai (CN); Xuemei Wang, Shanghai (CN)

(73) Assignee: Hoffmann-La Roche Inc., Little Falls, NJ (US)

(21) Appl. No.: 18/539,773

(22) Filed: Dec. 14, 2023

Related U.S. Application Data
(62) Division of application No. 16/828,408, filed on Mar. 24, 2020, now Pat. No. 11,873,302.

(30) Foreign Application Priority Data
Mar. 25, 2019 (WO) PCT/CN2019/079543

(51) Int. Cl. C07D 487/04

(52) U.S. Cl. CPC C07D 487/04

(57) ABSTRACT

Disclosed are novel solid forms of compound (I),

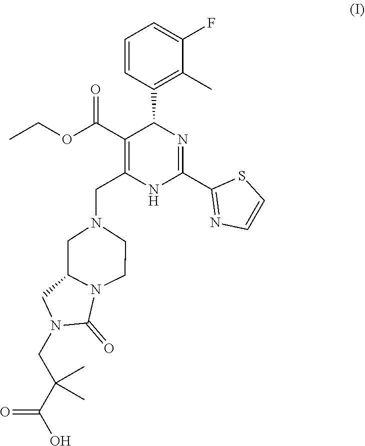 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

(3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid), and pharmaceutical compositions comprising solid forms thereof having characteristics described herein, which can be used as a HBV capsid inhibitor (or HBV Core Protein Allosteric Modifier), or for the treatment or prophylaxis of a viral disease in a patient relating to HBV infection or a disease caused by HBV infection.

### CROSS-REFERENCE TO RELATED APPLICATIONS

This application is a divisional of U.S. application Ser. No. 16/828,408, filed Mar. 24, 2020, now U.S. Pat. No. ______, which claims the benefit under 35 U.S.C. § 119(a) to International Application Serial No. PCT/CN2019/079543 filed Mar. 25, 2019, the contents of all of which are incorporated herein by reference in their entirety.

### TECHNICAL FIELD

The present invention relates to novel solid forms of compound (I),

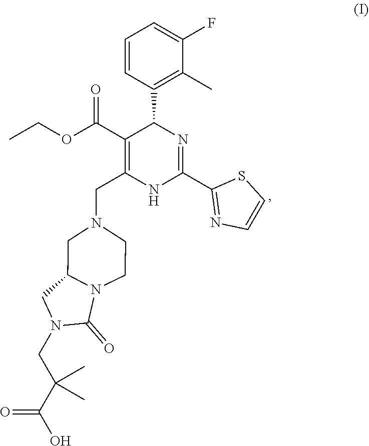 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

3-[(8aS)-7-[[(4S)-5-ethoxycarbonyl-4-(3-fluoro-2-methyl-phenyl)-2-thiazol-2-yl-1,4-dihydropyrimi din-6-yl]methyl]-3 -oxo-5,6,8,8a-tetrahydro-1H-imidazo[1,5-a]pyrazin-2-yl]-2,2-dimethyl-propanoic acid and pharmaceutical compositions comprising solid forms thereof disclosed herein, which can be used as a HBV capsid inhibitor (or HBV Core Protein Allosteric Modifier), or for the treatment or prophylaxis of a viral disease in a patient relating to HBV infection or a disease caused by HBV infection.

### BACKGROUND

HBV is a species of the hepadnaviridae family of viruses. HBV is a serious public health problem worldwide, with more than 400 million people especially in Asia-pacific regions chronically infected by this small enveloped DNA virus. Although most individuals seem to resolve the infection following acute symptoms, 15-40% of HBV patients will finally develop clinical diseases during their lifespan, most notably, hepatitis, liver cirrhosis, and hepatocellular carcinoma. Every year 500,000 to 1 million people die from the end stage of liver diseases caused by HBV infection.

HBV capsid protein plays essential roles in HBV replication. HBV has an icosahedral core comprising of 240 copies of the capsid (or core) protein. The predominant biological function of capsid protein is to act as a structural protein to encapsidate pre-genomic RNA and form immature capsid particles in the cytoplasm. This step is prerequisite for viral DNA replication. There has been a couple of capsid related anti-HBV inhibitors reported. For example, phenylpropenamide derivatives, including compounds named AT-61 and AT-130 (Feld J. et al. Antiviral Research 2007, 168-177), and a class of thiazolidin-4-ones from Valeant R&D (WO2006/033995), have been shown to inhibit pgRNA packaging. A recent study suggested that phenylpropenamides are, in fact, accelerators of HBV capsid assembly, and their actions result in the formation of empty capsids. These very interesting results illustrate the importance of the kinetic pathway in successful virus assembly.

Heteroaryldihydropyrimidines or HAP, including compounds named Bay 41-4109, Bay 38-7690 and Bay 39-5493, were discovered in a tissue culture-based screening (Deres K. et al. Science 2003, 893). These HAP analogs act as synthetic allosteric activators and are able to induce aberrant capsid formation that leads to degradation of the core protein. HAP analogs also reorganized core protein from preassembled capsids into noncapsid polymers, presumably by interaction of HAP with dimers freed during capsid ‘breathing’, the transitory breaking of individual intersubunit bonds. Bay 41-4109 was administered to HBV infected transgenic mouse or humanized mouse models and demonstrated in vivo efficacy with HBV DNA reduction (Deres K. et al. Science 2003, 893; Brezillon N. et al. PLoS ONE 2011, e25096). It was also shown that bis-ANS, a small molecule that acts as a molecular ‘wedge’ and interferes with normal capsid- protein geometry and capsid formation (Zlotnick A. et al. J. Virol. 2002, 4848-4854).

3-[(8aS)-7-[[(4S)-5-ethoxycarbonyl-4-(3-fluoro-2-methyl-phenyl)-2-thiazol-2-yl-1,4-dihydropyrimidin-6-yl] methyl]-3-oxo-5,6,8,8a-tetrahydro-1H-imidazo[1,5-a]pyrazin-2-yl]-2,2-dimethyl-propanoic acid (Compound (I)) was disclosed in WO2015/132276 as a HBV capsid inhibitor (or HBV Core Protein Allosteric Modifier).

It was found that Form D of compound (I) was physically unstable which leads to form change and makes it not suitable for further drug development. As one of the objectives of this patent, several novel solid forms were identified and characterized, showing significantly improved stability compared with Form D of compound (I). Developing novel forms of compound (I) with good processability or acceptable aqueous solubility is one of the objectives of current invention. Some novel solid forms enhance the developability of compound (I) fundamentally.

The present disclosure relates generally to the novel solid forms of compound (I) and processes to make them.

The physical stability of drug substances is an integral part of the systematic approach to the stability evaluation of pharmaceuticals due to its potential impacts on drug chemical stability performance and safety. The greater the stability is, the longer the shelf life could be. Therefore, the accelerated and long term stability testing used in this invention could be used to predict shelf lives.

Generally speaking, amorphous pharmaceuticals are markedly more soluble but less stable than their crystalline counterparts. In another embodiment, surprisingly, Form Amorphous of compound (I) significantly improved stability compared with Form D of compound (I).

In another embodiment, sodium salt Form J of compound (I) showed improved stability compared with Form D of compound (I) and improved solubility compared with some of other crystal forms of the parent compound (I). An in vivo PK study showed that Form J of compound (I) exhibited much slower absorption rate to reach Cmax. Therefore, sodium salt Form J is suitable to be formulated as sustained-release oral formulation. Although Form J converted to HC1 salt immediately, its apparent solubility in FaSSIF increased with time. Therefore, sodium salt Form J could be developed as enteric release formulations to avoid conversion in SGF and achieve higher solubility in intestinal environment for better absorption.

In another embodiment, Form H of compound (I) is a mono-hydrate which showed improved stability compared with Form D of compound (I). Generally speaking, hydrated crystal forms thermodynamically show the lowest solubility in water. Form H shows unexpected higher water solubility than Form A. With acceptable solid state stability, Form H of compound (I) is more preferred with oral suspension formulation.

### SUMMARY OF THE INVENTION

The present invention relates to polymorphs, salts, solvates, co-crystals or combinations thereof and methods for the synthesis and production of solid forms of 3-[(8aS)-7-[[(4S)-5-ethoxycarbonyl-4-(3-fluoro-2-methyl-phenyl)-2-thiazol-2-yl-1,4-dihydropyrimidin-6-yl] methyl]-3-oxo-5,6,8,8a-tetrahydro-1H-imidazo[1,5-a]pyrazin-2-yl]-2,2-dimethyl-propanoic acid.

One embodiment provided herein is an amorphous or solid form of compound (I) or solvates or combination thereof.

Another embodiment provided herein is an amorphous or solid form of compound (I), wherein the solid form is Form A, Form B, Form C, Form D, Form E, Form F, Form Form H, Form I, Form J, Form K, Form L, Form M, Form N, Form 0, Form P, Form Q, Form R, Form S, Form T, Form U, Form V, Form W, Form X, or a combination thereof.

In another embodiment, the solid form of compound (I) is Form D that exhibits an X-ray powder diffraction (XRPD) pattern with characteristic peaks expressed in degrees 2-theta at 6.8°±0.2°, 13.0°±0.2°, 20.3°±0.2°, 27.1°±0.2°, 27.4°±0.2°, 28.8°±0.2° and 29.1°±0.2°.

In a further embodiment, the solid form of compound (I) is Form D that exhibits an X-ray powder diffraction (XRPD) pattern shown in FIG. 1.

In another embodiment, the solid form of compound (I) is Form A that exhibits an X-ray powder diffraction (XRPD) pattern with characteristic peaks expressed in degrees 2-theta at 10.0°±0.2°, 14.5°±0.2°, 15.4°±0.2°, 16.4°±0.2°, 19.4°±0.2°, 21.1°±0.2° and 23.2°±0.2°.

In a further embodiment, the solid form of compound (I) is Form A that exhibits an X-ray powder diffraction (XRPD) pattern with characteristic peaks expressed in degrees 2-theta at 10.0°±0.2°, 12.3°±0.2°, 13.2°±0.2°, 14.5°±0.2°, 15.4°±0.2°, 16.4°±0.2°, 19.4°±0.2°, 20.3°±0.2°, 21.1°±0.2°, 21.6°±0.2°, 23.2°±0.2°, 23.7°±0.2°, 24.5°±0.2°, 25.5°±0.2° and 26.8°±0.2°.

In a further embodiment, the solid form of compound (I) is Form A that exhibits an X-ray powder diffraction (XRPD) pattern shown in FIG. 2.

In another embodiment, the solid form of compound (I) is Form Amorphous that exhibits an X-ray powder diffraction (XRPD) pattern shown in FIG. 4.

In another embodiment, the solid form of compound (I) is Form B that exhibits an X-ray powder diffraction (XRPD) pattern with characteristic peaks expressed in degrees 2-theta at 3.9°±0.2°, 4.8°±0.2°, 7.3°±0.2°, 7.8°±0.2°, 10.7°±0.2°, 15.6°±0.2° and 19.5°±0.2°.

In a further embodiment, the solid form of compound (I) is Form B that exhibits an X-ray powder diffraction (XRPD) pattern with characteristic peaks expressed in degrees 2-theta at 3.9°±0.2°, 4.8°±0.2°, 7.3°±0.2°, 7.8°±0.2°, 10.7°±0.2°, 15.6°±0.2°, 16.2°±0.2°, 16.4°±0.2°, 19.5°±0.2°, 20.4°±0.2° and 21.7°±0.2°.

In a further embodiment, the solid form of compound (I) is Form B that exhibits an X-ray powder diffraction (XRPD) pattern shown in FIG. 5.

In another embodiment, the solid form of compound (I) is Form C that exhibits an X-ray powder diffraction (XRPD) pattern with characteristic peaks expressed in degrees 2-theta at 5.1°±0.2°, 10.6°±0.2°, 10.8°±0.2°, 12.1°±0.2°, 13.6°±0.2° and 13.9°±0.2°.

In a further embodiment, the solid form of compound (I) is Form C that exhibits an X-ray powder diffraction (XRPD) pattern shown in FIG. 6.

In another embodiment, the solid form of compound (I) is Form E that exhibits an X-ray powder diffraction (XRPD) pattern with characteristic peaks expressed in degrees 2-theta at 4.0°±0.2°, 5.1°±0.2°, 5.4°±0.2°, 10.2°±0.2°, 13.3°±0.2°, 15.5°±0.2° and 20.2°±0.2° .

In a further embodiment, the solid form of compound (I) is Form E that exhibits an X-ray powder diffraction (XRPD) pattern with characteristic peaks expressed in degrees 2-theta at 4.0°±0.2°, 5.1°±0.2°, 5.4°±0.2°, 10.2°±0.2°, 10.5°±0.2°, 11.8°±0.2°, 12.2°±0.2°, 13.3°±0.2°, 13.8°±0.2°, 14.6°±0.2°, 15.5°±0.2°, 15.8°±0.2°, 16.5°±0.2°, 19.5°±0.2°, 20.2°±0.2° and 21.9°±0.2°.

In a further embodiment, the solid form of compound (I) is Form E that exhibits an X-ray powder diffraction (XRPD) pattern shown in FIG. 7.

In another embodiment, the solid form of compound (I) is Form F that exhibits an X-ray powder diffraction (XRPD) pattern with characteristic peaks expressed in degrees 2-theta at 4.0°±0.2°, 4.9°±0.2°, 7.1°±0.2°, 15.8°±0.2°, 20.3°±0.2° and 21.9°±0.2°.

In a further embodiment, the solid form of compound (I) is Form F that exhibits an X-ray powder diffraction (XRPD) pattern with characteristic peaks expressed in degrees 2-theta at 4.0°±0.2°, 4.9°±0.2°, 7.1°±0.2°, 7.4°±0.2°, 7.9°±0.2°, 10.6°±0.2°, 11.9°±0.2°, 13.1°±0.2°, 13.3°±0.2°, 13.8°±0.2°, 15.8°±0.2°, 20.3°±0.2°, 21.0°±0.2° and 21.9°±0.2°.

In a further embodiment, the solid form of compound (I) is Form F that exhibits an X-ray powder diffraction (XRPD) pattern shown in FIG. 8.

In another embodiment, the solid form of compound (I) is Form G that exhibits an X-ray powder diffraction (XRPD) pattern with characteristic peaks expressed in degrees 2-theta at 3.7°±0.2°, 4.1°±0.2°, 5.0°±0.2°, 6.2°±0.2°, 7.7°±0.2°, 8.2°±0.2° and 17.1°±0.2°.

In a further embodiment, the solid form of compound (I) is Form G that exhibits an X-ray powder diffraction (XRPD) pattern with characteristic peaks expressed in degrees 2-theta at 3.7°±0.2°, 4.1°±0.2°, 5.0°±0.2°, 6.2°±0.2°, 7.7°±0.2°, 8.2°±0.2°, 11.3°±0.2°, 13.3°±0.2°, 13.8°±0.2°, 14.5°±0.2°, 16.3°±0.2°, 17.1°±0.2°, 19.3°±0.2°, 21.1°±0.2° and 23.3°±0.2°.

In a further embodiment, the solid form of compound (I) is Form G that exhibits an X-ray powder diffraction (XRPD) pattern shown in FIG. 9.

In another embodiment, the solid form of compound (I) is Form J that exhibits an X-ray powder diffraction (XRPD) pattern with characteristic peaks expressed in degrees 2-theta at 7.7°±0.2°, 9.7°±0.2°, 14.7°±0.2°, 15.9°±0.2°, 22.0°±0.2°, 23.4°±0.2°.

In a further embodiment, the solid form of compound (I) is Form J that exhibits an X-ray powder diffraction (XRPD) pattern with characteristic peaks expressed in degrees 2-theta at 7.7°±0.2°, 9.7°±0.2°, 11.5°±0.2°, 13.0°±0.2°, 14.7°±0.2°, 15.3°±0.2°, 15.9°±0.2°, 16.5°±0.2°, 19.0°±0.2°, 22.0°±0.2°, 22.6°±0.2°, 23.4°±0.2°, 23.9°±0.2°, 24.5°±0.2° and 25.3°±0.2°.

In a further embodiment, the solid form of compound (I) is Form J that exhibits an X-ray powder diffraction (XRPD) pattern shown in FIG. 10.

In a further embodiment, the solid form of compound (I) is Form J, wherein the Form J is the sodium salt of compound (I).

In another embodiment, the solid form of compound (I) is Form H that exhibits an X-ray powder diffraction (XRPD) pattern with characteristic peaks expressed in degrees 2-theta at 8.0°±0.2°, 9.7°±0.2°, 14.6°±0.2°, 15.7°±0.2°, 15.9°±0.2° and 24.1°±0.2°.

In a further embodiment, the solid form of compound (I) is Form H that exhibits an X-ray powder diffraction (XRPD) pattern with characteristic peaks expressed in degrees 2-theta at 6.8°±0.2°, 8.0°±0.2°, 9.7°±0.2°, 11.6°±0.2°, 14.6°±0.2°, 15.2°±0.2°, 15.7°±0.2°, 15.9°±0.2°, 18.9°±0.2°, 19.9°±0.2°, 22.7°±0.2°, 24.1°±0.2°, 24.5°±0.2° and 26.0°±0.°.

In a further embodiment, the solid form of compound (I) is Form H that exhibits an X-ray powder diffraction (XRPD) pattern shown in FIG. 12.

In another embodiment, the solid form of compound (I) is Form I that exhibits an X-ray powder diffraction (XRPD) pattern with characteristic peaks expressed in degrees 2-theta at 6.4°±0.2°, 7.8°±0.2°, 9.9°±0.2°, 11.6°±0.2°, 16.2°±0.2° and 22.1°±0.2°.

In a further embodiment, the solid form of compound (I) is Form I that exhibits an X-ray powder diffraction (XRPD) pattern with characteristic peaks expressed in degrees 2-theta at 6.4°±0.2°, 7.8°±0.2°, 9.6°±0.2°, 9.9°±0.2°, 11.6°±0.2°, 13.0°±0.2°, 14.5°±0.2°, 15.0°±0.2°, 15.7°±0.2°, 16.2°±0.2°, 18.3°±0.2°, 22.1°±0.2°, 23.0°±0.2°, 24.3°±0.2° and 27.2°±0.2°.

In a further embodiment, the solid form of compound (I) is Form I that exhibits an X-ray powder diffraction (XRPD) pattern shown in FIG. 15.

In another embodiment, the solid form of compound (I) is Form K that exhibits an X-ray powder diffraction (XRPD) pattern with characteristic peaks expressed in degrees 2-theta at 5.4°±0.2°, 13.3°±0.2°, 15.9°±0.2°, 16.3°±0.2°, 18.0°±0.2° and 22.7°±0.2°.

In a further embodiment, the solid form of compound (I) is Form K that exhibits an X-ray powder diffraction (XRPD) pattern with characteristic peaks expressed in degrees 2-theta at 5.4°±0.2°, 13.3°±0.2°, 13.8°±0.2°, 14.8°±0.2°, 15.9°±0.2°, 16.3°±0.2°, 18.0°±0.2°, 19.5°±0.2°, 20.0°±0.2°, 21.7°±0.2°, 22.4°±0.2°, 22.7°±0.2°, 23.4°±0.2°, 24.1°±0.2° and 28.0°±0.2°.

In a further embodiment, the solid form of compound (I) is Form K that exhibits an X-ray powder diffraction (XRPD) pattern shown in FIG. 16.

In a further embodiment, the solid form of compound (I) is Form K, wherein the Form K is the hydrochloride salt of compound (I).

In another embodiment, the solid form of compound (I) is Form L that exhibits an X-ray powder diffraction (XRPD) pattern with characteristic peaks expressed in degrees 2-theta at 6.0°±0.2°, 11.8°±0.2°, 15.3°±0.2°, 15.8°±0.2°, 18.3°±0.2° and 24.4°±0.2°.

In a further embodiment, the solid form of compound (I) is Form L that exhibits an X-ray powder diffraction (XRPD) pattern with characteristic peaks expressed in degrees 2-theta at 6.0°±0.2°, 11.2°±0.2°, 11.8°±0.2°, 12.3°±0.2°, 13.1°±0.2°, 15.3°±0.2°, 15.8°±0.2°, 18.3°±0.2°, 18.7°±0.2°, 21.7°±0.2°, 22.5°±0.2°, 23.8°±0.2°, 24.4°±0.2°, 25.7°±0.2° and 27.7°±0.2°.

In a further embodiment, the solid form of compound (I) is Form L that exhibits an X-ray powder diffraction (XRPD) pattern shown in FIG. 17.

In a further embodiment, the solid form of compound (I) is Form L, wherein the Form L is the hydrochloride salt of compound (I).

In another embodiment, the solid form of compound (I) is Form M that exhibits an X-ray powder diffraction (XRPD) pattern with characteristic peaks expressed in degrees 2-theta at 5.3°±0.2°, 7.7°±0.2°, 10.7°±0.2°, 17.6°±0.2°, 19.0°±0.2° and 19.2°±0.2°.

In a further embodiment, the solid form of compound (I) is Form M that exhibits an X-ray powder diffraction (XRPD) pattern with characteristic peaks expressed in degrees 2-theta at 5.3°±0.2°, 7.7°±0.2°, 9.4°±0.2°, 10.7°±0.2°, 15.5°±0.2°, 17.2°±0.2°, 17.6°±0.2°, 19.0°±0.2°, 19.2°±0.2°, 19.8°±0.2° and 24.4°±0.2°.

In a further embodiment, the solid form of compound (I) is Form M that exhibits an X-ray powder diffraction (XRPD) pattern shown in FIG. 19.

In a further embodiment, the solid form of compound (I) is Form M, wherein the Form M is the sulfate salt of compound (I).

In another embodiment, the solid form of compound (I) is Form N that exhibits an X-ray powder diffraction (XRPD) pattern with characteristic peaks expressed in degrees 2-theta at 5.3°±0.2°, 10.7°±0.2°, 18.0°±0.2°, 18.7°±0.2°, 19.4°±0.2°, 20.3°±0.2°, 21.5°±0.2° and 24.7°±0.2°.

In a further embodiment, the solid form of compound (I) is Form N that exhibits an X-ray powder diffraction (XRPD) pattern shown in FIG. 20.

In a further embodiment, the solid form of compound (I) is Form N, wherein the Form N is the sulfate salt of compound (I).

In another embodiment, the solid form of compound (I) is Form O that exhibits an X-ray powder diffraction (XRPD) pattern with characteristic peaks expressed in degrees 2-theta at 4.9°±0.2°, 10.6°±0.2°, 14.3°±0.2°, 22.4°±0.2° and 22.9°±0.2°.

In a further embodiment, the solid form of compound (I) is Form O that exhibits an X-ray powder diffraction (XRPD) pattern with characteristic peaks expressed in degrees 2-theta at 4.9°±0.2°, 10.6°±0.2°, 13.2°±0.2°, 14.3°±0.2°, 16.9°±0.2°, 17.9°±0.2°, 19.1°±0.2°, 20.2°±0.2°, 21.1°±0.2°, 22.4°±0.2°, 22.9°±0.2°, 23.9°±0.2° and 24.4°±0.2°.

In a further embodiment, the solid form of compound (I) is Form O that exhibits an X-ray powder diffraction (XRPD) pattern shown in FIG. 21.

In a further embodiment, the solid form of compound (I) is Form O, wherein the Form O is the besylate salt of compound (I).

In another embodiment, the solid form of compound (I) is Form P that exhibits an X-ray powder diffraction (XRPD) pattern with characteristic peaks expressed in degrees 2-theta at 3.9°±0.2°, 7.7°±0.2°, 15.3°±0.2°, 21.5°±0.2°, 27.5°±0.2° and 31.8°±0.2°.

In a further embodiment, the solid form of compound (I) is Form P that exhibits an X-ray powder diffraction (XRPD) pattern shown in FIG. 22.

In a further embodiment, the solid form of compound (I) is Form P, wherein the Form P is the potassium salt of compound (I).

In another embodiment, the solid form of compound (I) is Form Q that exhibits an X-ray powder diffraction (XRPD) pattern with characteristic peaks expressed in degrees 2-theta at 7.9°±0.2°, 8.7°±0.2°, 13.2°±0.2°, 15.4°±0.2°, 21.8°±0.2°, 26.3°±0.2° and 29.3°±0.2°.

In a further embodiment, the solid form of compound (I) is Form Q that exhibits an X-ray powder diffraction (XRPD) pattern with characteristic peaks expressed in degrees 2-theta at 7.9°±0.2°, 8.7°±0.2°, 10.5°±0.2°, 11.0°±0.2°, 13.2°±0.2°, 15.4°±0.2°, 16.8°±0.2°, 17.4°±0.2°, 18.1°±0.2°, 18.5°±0.2°, 21.2°±0.2°, 21.8°±0.2°, 26.3°±0.2°, 26.7°±0.2° and 29.3°±0.2°.

In a further embodiment, the solid form of compound (I) is Form Q that exhibits an X-ray powder diffraction (XRPD) pattern shown in FIG. 23.

In a further embodiment, the solid form of compound (I) is Form Q, wherein the Form Q is the potassium salt of compound (I).

In another embodiment, the solid form of compound (I) is Form R that exhibits an X-ray powder diffraction (XRPD) pattern with characteristic peaks expressed in degrees 2-theta at 7.5°±0.2°, 7.8°±0.2°, 9.9°±0.2°, 14.8°±0.2°, 15.4°±0.2°, 15.7°±0.2° and 22.2°±0.2°.

In a further embodiment, the solid form of compound (I) is Form R that exhibits an X-ray powder diffraction (XRPD) pattern with characteristic peaks expressed in degrees 2-theta at 7.5°±0.2°, 7.8°±0.2°, 8.8°±0.2°, 9.9°±0.2°, 11.2°±0.2°, 11.7°±0.2°, 12.4°±0.2°, 14.8°±0.2°, 15.4°±0.2°, 15.7°±0.2°, 17.2°±0.2°, 22.2°±0.2° and 26.3°±0.2°.

In a further embodiment, the solid form of compound (I) is Form R that exhibits an X-ray powder diffraction (XRPD) pattern shown in FIG. 24.

In a further embodiment, the solid form of compound (I) is Form R, wherein the Form R is the potassium salt of compound (I).

In another embodiment, the solid form of compound (I) is Form S that exhibits an X-ray powder diffraction (XRPD) pattern with characteristic peaks expressed in degrees 2-theta at 8.3°±0.2°, 8.7°±0.2°, 13.7°±0.2°, 15.8°±0.2°, 18.0°±0.2° and 21.7°±0.2°.

In a further embodiment, the solid form of compound (I) is Form S that exhibits an X-ray powder diffraction (XRPD) pattern with characteristic peaks expressed in degrees 2-theta at 8.3°±0.2°, 8.7°±0.2°, 11.0°±0.2°, 11.2°±0.2°, 13.4°±0.2°, 13.7°±0.2°, 15.8°±0.2°, 16.6°±0.2°, 18.0°±0.2°, 20.9°±0.2°, 21.7°±0.2°, 24.5°±0.2°, 26.2°±0.2°, 26.7°±0.2° and 28.6°±0.2°.

In a further embodiment, the solid form of compound (I) is Form S that exhibits an X-ray powder diffraction (XRPD) pattern shown in FIG. 25.

In a further embodiment, the solid form of compound (I) is Form S, wherein the Form S is the potassium salt of compound (I).

In another embodiment, the solid form of compound (I) is Form T that exhibits an X-ray powder diffraction (XRPD) pattern with characteristic peaks expressed in degrees 2-theta at 8.0°±0.2°, 10.8°±0.2°, 11.1°±0.2°, 13.3°±0.2°, 15.5°±0.2°, 21.5°±0.2° and 31.6°±0.2°.

In a further embodiment, the solid form of compound (I) is Form T that exhibits an X-ray powder diffraction (XRPD) pattern shown in FIG. 26.

In a further embodiment, the solid form of compound (I) is Form T, wherein the Form T is the calcium salt of compound (I).

In another embodiment, the solid form of compound (I) is Form U that exhibits an X-ray powder diffraction (XRPD) pattern with characteristic peaks expressed in degrees 2-theta at 7.5°±0.2°, 10.1°±0.2°, 10.6°±0.2°, 13.7°±0.2°, 18.9°±0.2°, 20.3°±0.2° and 21.0°±0.2°.

In a further embodiment, the solid form of compound (I) is Form U that exhibits an X-ray powder diffraction (XRPD) pattern with characteristic peaks expressed in degrees 2-theta at 7.5°±0.2°, 9.6°±0.2°, 10.1°±0.2°, 10.6°±0.2°, 11.9°±0.2°, 12.6°±0.2°, 12.9°±0.2°, 13.7°±0.2°, 16.2°±0.2°, 17.8°±0.2°, 18.9°±0.2°, 20.3°±0.2° and 21.0°±0.2°.

In a further embodiment, the solid form of compound (I) is Form U that exhibits an X-ray powder diffraction (XRPD) pattern shown in FIG. 27.

In a further embodiment, the solid form of compound (I) is Form U, wherein the Form U is the calcium salt of compound (I).

In another embodiment, the solid form of compound (I) is Form V that exhibits an X-ray powder diffraction (XRPD) pattern with characteristic peaks expressed in degrees 2-theta at 5.6°±0.2°, 8.5°±0.2°, 14.2°±0.2°, 16.2°±0.2°, 21.9°±0.2° and 22.4°±0.2°.

In a further embodiment, the solid form of compound (I) is Form V that exhibits an X-ray powder diffraction (XRPD) pattern shown in FIG. 28.

In a further embodiment, the solid form of compound (I) is Form V, wherein the Form V is the ammonium salt of compound (I).

In another embodiment, the solid form of compound (I) is Form W that exhibits an X-ray powder diffraction (XRPD) pattern with characteristic peaks expressed in degrees 2-theta at 6.2°±0.2°, 7.5°±0.2°, 7.8°±0.2°, 11.4°±0.2°, 15.8°±0.2° and 21.4°±0.2°.

In a further embodiment, the solid form of compound (I) is Form W that exhibits an X-ray powder diffraction (XRPD) pattern with characteristic peaks expressed in degrees 2-theta at 6.2°±0.2°, 6.6°±0.2°, 7.5°±0.2°, 7.8°±0.2°, 9.5°±0.2°, 9.8°±0.2°, 11.4°±0.2°, 12.5°±0.2°, 13.5°±0.2°, 14.5°±0.2°, 15.8°±0.2°, 19.8°±0.2°, 21.4°±0.2°, 22.5°±0.2°, 24.0°±0.2° and 26.5°±0.2°.

In a further embodiment, the solid form of compound (I) is Form W that exhibits an X-ray powder diffraction (XRPD) pattern shown in FIG. 29.

In a further embodiment, the solid form of compound (I) is Form W, wherein the Form W is the ammonium salt of compound (I).

In another embodiment, the solid form of compound (I) is Form X that exhibits an X-ray powder diffraction (XRPD) pattern with characteristic peaks expressed in degrees 2-theta at 8.6°±0.2°, 11.1°±0.2°, 11.4°±0.2°, 14.3°±0.2°, 16.0°±0.2°, 16.3°±0.2° and 22.0°±0.2°.

In a further embodiment, the solid form of compound (I) is Form X that exhibits an X-ray powder diffraction (XRPD) pattern with characteristic peaks expressed in degrees 2-theta at 7.6°±0.2°, 8.6°±0.2°, 11.1°±0.2°, 11.4°±0.2°, 12.6°±0.2°, 14.3°±0.2°, 16.0°±0.2°, 16.3°±0.2°, 19.8°±0.2°, 21.5°±0.2°, 22.0°±0.2° and 23.2°±0.2°.

In a further embodiment, the solid form of compound (I) is Form X that exhibits an X-ray powder diffraction (XRPD) pattern shown in FIG. 30.

In a further embodiment, the solid form of compound (I) is Form X, wherein the Form X is the ammonium salt of compound (I).

Another embodiment provided herein is a pharmaceutical composition comprising the solid forms disclosed herein and a pharmaceutically acceptable carrier, excipient, diluent, adjuvant, vehicle, or a combination thereof.

Another embodiment provided herein is the use of the solid form disclosed herein or the pharmaceutical composition for the manufacture of a medicament for the treatment or prophylaxis of a viral disease in a patient.

In another embodiment, the viral disease disclosed herein is HBV infection or a disease caused by HBV infection.

Another embodiment provided herein is a method for the treatment or prophylaxis of HBV infection or a disease caused by HBV infection, which method comprises administering a therapeutically effective amount of the solid form or the pharmaceutical composition disclosed herein.

### ABBREVIATIONS

ACN Acetonitrile

Cmax Maximum concentration observed

DSC Differential Scanning calorimetry

EtOAc Ethyl acetate

FaSSIF Fasted State Simulated Intestinal Fluid

IPA Isopropanol

IPAc Isopropyl acetate

IPE Diisopropyl ether

Pos. Position

Rel. Int. Relative Intensity

RT Room temperature

SGF Simulated Gastric Fluid

TGA Thermal Gravimetric Analysis

Tmax Time at which the maximum concentration (Cmax) is observed

XRPD X-ray powder diffraction

### DESCRIPTION OF THE FIGURES

FIG. 1 X-ray powder diffraction pattern for Form D

FIG. 2 X-ray powder diffraction pattern for Form A

FIG. 3 X-ray crystal structure of Form A

FIG. 4 X-ray powder diffraction pattern for Form Amorphous

FIG. 5 X-ray powder diffraction pattern for Form B

FIG. 6 X-ray powder diffraction pattern for Form C

FIG. 7 X-ray powder diffraction pattern for Form E

FIG. 8 X-ray powder diffraction pattern for Form F

FIG. 9 X-ray powder diffraction pattern for Form G

FIG. 10 X-ray powder diffraction pattern for of sodium salt Form J

FIG. 11 X-ray crystal structure of sodium salt Form J

FIG. 12 X-ray powder diffraction pattern for Form H

FIG. 13 DSC thermogram of Form H

FIG. 14 TGA diagram of Form H

FIG. 15 X-ray powder diffraction pattern for Form I

FIG. 16 X-ray crystal structure of HCl salt Form K

FIG. 17 X-ray powder diffraction pattern for HCl salt Form L

FIG. 18 X-ray crystal structure of HCl salt Form L

FIG. 19 X-ray powder diffraction pattern for H₂SO₄ salt Form M

FIG. 20 X-ray powder diffraction pattern for H₂SO₄ salt Form N

FIG. 21 X-ray powder diffraction pattern for besylate salt Form O

FIG. 22 X-ray powder diffraction pattern for potassium salt Form P

FIG. 23 X-ray powder diffraction pattern for potassium salt Form Q

FIG. 24 X-ray powder diffraction pattern for potassium salt Form R

FIG. 25 X-ray powder diffraction pattern for potassium salt Form S

FIG. 26 X-ray powder diffraction pattern for calcium salt Form T

FIG. 27 X-ray powder diffraction pattern for calcium salt Form U

FIG. 28 X-ray powder diffraction pattern for ammonium salt Form V

FIG. 29 X-ray powder diffraction pattern for ammonium salt Form W

FIG. 30 X-ray powder diffraction pattern for ammonium salt Form X

### EXAMPLES

The invention will be more fully understood by reference to the following examples. They should not, however, be construed as limiting the scope of the invention.

### HPLC Method for Chemical Purity and Assay Test

HPLC condition is disclosed here in Table 1.

TABLE 1
HPLC conditions for chemical purity and assay test

| Instrument | Agilent 1260 HPLC system | | |
|---|---|---|---|
| Column | Waters Xbridge C8 (4.6 × 150 mm × 3.5 μm) | | |
| Oven temperature | 30° C. | | |
| Mobile phase | A: 0.12% TFA in water | | |
| | B: 0.12% TFA in ACN | | |
| Gradient program | Time (min) | A % | B % |
| | 0.00 | 80 | 20 |
| | 15.00 | 50 | 50 |
| | 20.00 | 10 | 90 |
| | 25.00 | 10 | 90 |
| | 25.01 | 80 | 20 |
| | 30.00 | 80 | 20 |

| Flow rate | 1.0 | mL/min |
|---|---|---|
| Detector | UV 299 | nm |
| Nominal concentration | 0.5 | mg/mL |
| Diluent | ACN:water, 1:1 | |
| Injection volume | 10 | μL |
| Retention Time | ~12.7 | min |

### Example 1: Preparation of Form D of Compound (I)

A solution of 10 mg of compound (I) in 5 mL n-propanol was placed at room temperature and evaporated to dryness.

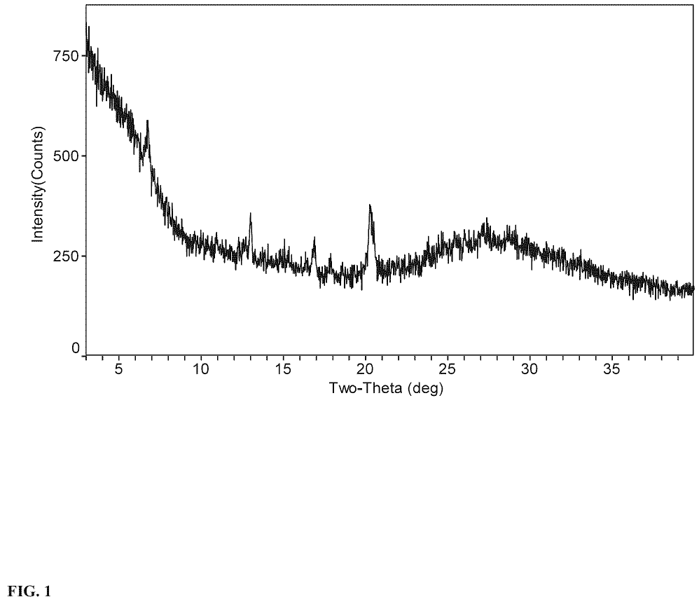

Solids were obtained and characterized by XRPD. The XRPD pattern of Form D of compound (I) is shown in FIG. 1. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 2
X-ray powder diffraction peaks of Form D of compound (I)

| Pos. [°2-theta] | Rel. Int. [%] |
|---|---|
| 6.8 | 100 |
| 13.0 | 61 |
| 15.3 | 46 |
| 16.9 | 50 |
| 20.3 | 64 |
| 27.1 | 56 |
| 27.4 | 56 |
| 28.8 | 55 |
| 29.1 | 55 |

### Example 2: Alternative Preparation of Form D of Compound (I)

A solution of 10 mg of compound (I) in 5 mL a mixture of n-propanol and 2-butanol (2:8, v:v) was placed at room temperature and evaporated to dryness.

The solid was collected for XRPD analysis. The XRPD pattern of the solid was the same as that in Table 2 and confirmed to be Form D of compound (I).

### Example 3: Preparation of Form A of Compound (I)

A solution of 10 mg of compound (I) in 10 mL acetone was placed at room temperature and evaporated to dryness.

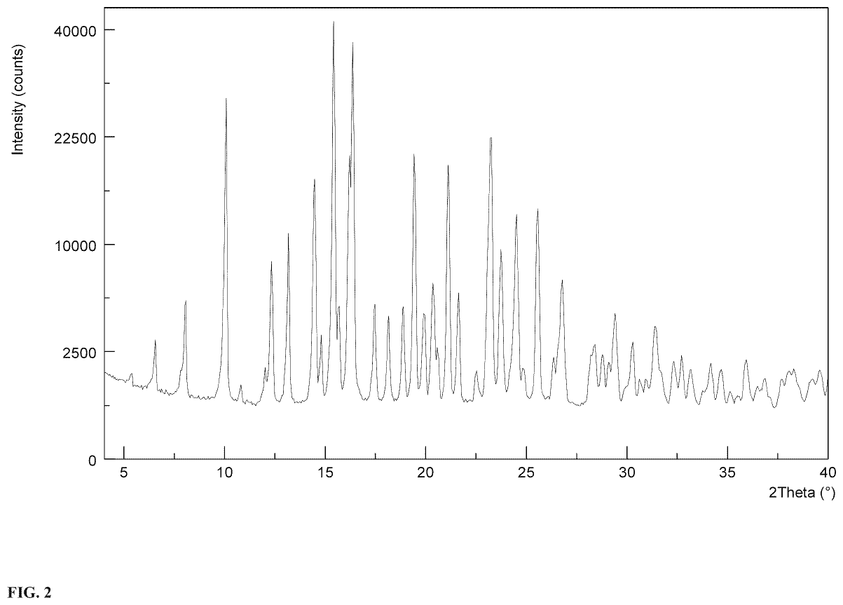

The XRPD pattern of Form A of compound (I) is shown in FIG. 2. Major peaks and their related intensities in the XRPD pattern are shown in table below.

### Experimental Conditions

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.053° at a scanning speed of 10.504°/min.

TABLE 3
X-ray powder diffraction peaks of Form A of compound (I)

| Pos. [°2-theta] | Rel. Int. [%] |
|---|---|
| 5.3 | 1 |
| 6.5 | 4 |
| 8.0 | 12 |
| 10.0 | 64 |
| 10.8 | 1 |
| 12.3 | 17 |
| 13.2 | 22 |
| 14.5 | 40 |
| 14.8 | 6 |
| 15.4 | 100 |
| 16.4 | 89 |
| 17.4 | 11 |
| 18.1 | 9 |
| 18.9 | 11 |
| 19.4 | 46 |
| 19.9 | 10 |
| 20.3 | 14 |
| 21.1 | 45 |
| 21.6 | 13 |
| 22.5 | 2 |
| 23.2 | 54 |
| 23.7 | 21 |
| 24.5 | 29 |
| 25.5 | 31 |
| 26.8 | 15 |
| 28.4 | 5 |
| 28.8 | 4 |
| 29.4 | 9 |
| 30.3 | 5 |
| 31.4 | 7 |
| 32.3 | 3 |
| 32.7 | 4 |
| 33.1 | 3 |
| 34.2 | 3 |
| 34.7 | 2 |
| 35.9 | 3 |
| 36.8 | 2 |
| 37.7 | 2 |
| 38.2 | 2 |
| 39.2 | 1 |
| 39.6 | 1 |

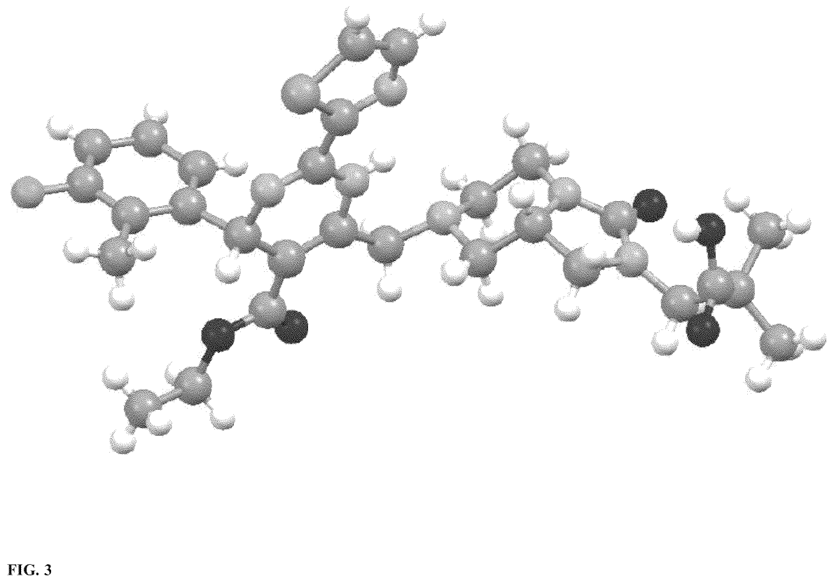

FIG. 3 shows the X-ray structure of Form A. The single crystal X-ray intensity data were collected at 296 K using a Bruker SMART APEX II with Cu-K-alpha-radiation (1.54 Å). Structure solution and refinement was performed using the She1XTL software (Bruker AXS, Karlsruhe). The crystal data and structure refinement is shown in Table 4.

TABLE 4
Single crystal structural data of Forms A

| Crystal form | Form A | |
|---|---|---|
| Solid form description | Polymorph | |
| Measuring Temperature | 296 K | |
| Crystal system | Monoclinic | |
| Space group | P 2₁ | |
| Unit cell dimensions | 7.4967(2) | Å |
| a= | | |
| b= | 12.1773(2) | Å |
| c= | 18.5541(4) | Å |
| α= | 90° | |
| β= | 94.4110(10)° | |
| γ= | 90° | |
| Cell volume | 1688.78(6) | Å³ |
| API molecules in unit cell | 4 | |
| Calculated density | 1.386 | g/cm³ |

### Example 4: Alternative Preparation of Form A of Compound (I)

A solution of 10 mg of compound (I) in 1 mL ethyl acetate was placed at room temperature and evaporated to dryness.

The solid was collected for XRPD analysis. The XRPD pattern of the solid was the same as that in Table 3 and confirmed to be Form A of compound (I).

### Example 5: Alternative Preparation of Form A of Compound (I)

A solution of 10 mg of compound (I) in 2 mL isopropyl acetate was placed at room temperature and evaporated to dryness.

The solid was collected for XRPD analysis. The XRPD pattern of the solid was the same as that in Table 3 and confirmed to be Form A of compound (I).

### Example 6: Alternative Preparation of Form A of Compound (I)

A solution of 10 mg of compound (I) in 4 mL acetonitrile was placed at room temperature and evaporated to dryness.

The solid was collected for XRPD analysis. The XRPD pattern of the solid was the same as that in Table 3 and confirmed to be Form A of compound (I).

### Example 7: Preparation of Form Amorphous of Compound (I)

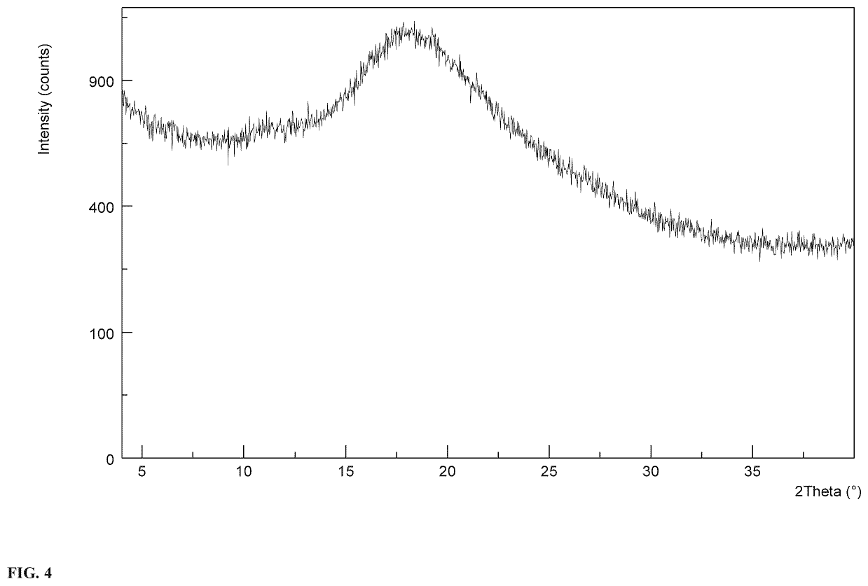

A solution of 500 mg of compound (I) in 10 mL dichloromethane was rapidly evaporated using a rotary evaporator. The solid was dried at 30° C. overnight. The solid was analyzed by XRPD. The result is shown in FIG. 4.

### Characterization Method

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.053° at a scanning speed of 10.504°/min.

### Example 8: Alternative Preparation of Form Amorphous of Compound (I)

A solution of 10 mg of compound (I) in 1 mL methanol was placed at room temperature and evaporated to dryness.

The solid was collected for XRPD analysis. The XRPD pattern of the solid was the same as that in FIG. 4 and confirmed to be Form Amorphous of compound (I).

### Example 9: Alternative Preparation of Form Amorphous of Compound (I)

A solution of 10 mg of compound (I) in 1 mL mixture solvents of methanol and dichloromethane (50:50, v:v) was placed at room temperature and evaporated to dryness.

The solid was collected for XRPD analysis. The XRPD pattern of the solid was the same as that in FIG. 4. and confirmed to be Form Amorphous of compound (I).

### Example 10: Preparation of Form B of Compound (I)

Approximate 50 mg of Form Amorphous of compound (I) as prepared in Example 7 was weighed and transferred to a glass vial. 0.4 mL ethanol was added to form a suspension. The vial was mounted to a shaker and kept shaking at 25° C. with 1200 rpm for 3 min.

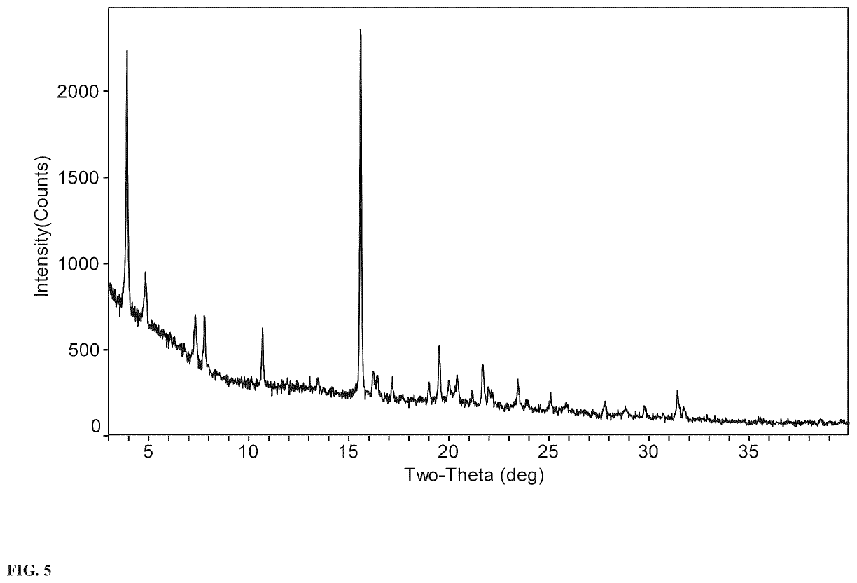

The XRPD pattern of Form B of compound (I) is shown in FIG. 5. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 5
X-ray powder diffraction peaks of Form B of compound (I)

| Pos. [°2-theta] | Rel. Int. [%] |
|---|---|
| 3.9 | 95 |
| 4.8 | 40 |
| 7.3 | 30 |
| 7.8 | 29 |
| 10.7 | 26 |
| 11.9 | 14 |
| 15.6 | 100 |
| 16.2 | 16 |
| 16.4 | 15 |
| 17.2 | 14 |
| 19.0 | 13 |
| 19.5 | 22 |
| 20.0 | 13 |
| 20.4 | 15 |
| 21.7 | 17 |
| 22.0 | 11 |
| 23.5 | 14 |
| 23.9 | 9 |
| 25.1 | 10 |
| 25.9 | 8 |
| 27.8 | 8 |
| 28.8 | 7 |
| 29.8 | 7 |
| 31.4 | 11 |
| 31.7 | 7 |
| 35.5 | 5 |

### Example 11: Preparation of Form C of Compound (I)

Approximate 50 mg of Form Amorphous of compound (I) as prepared in Example 7 was weighed and transferred to a glass vial. 0.5 mL a mixture of ethanol and methyl cyclohexane (1:4, v:v) was added to form a suspension. The suspension was agitated for 10 minutes.

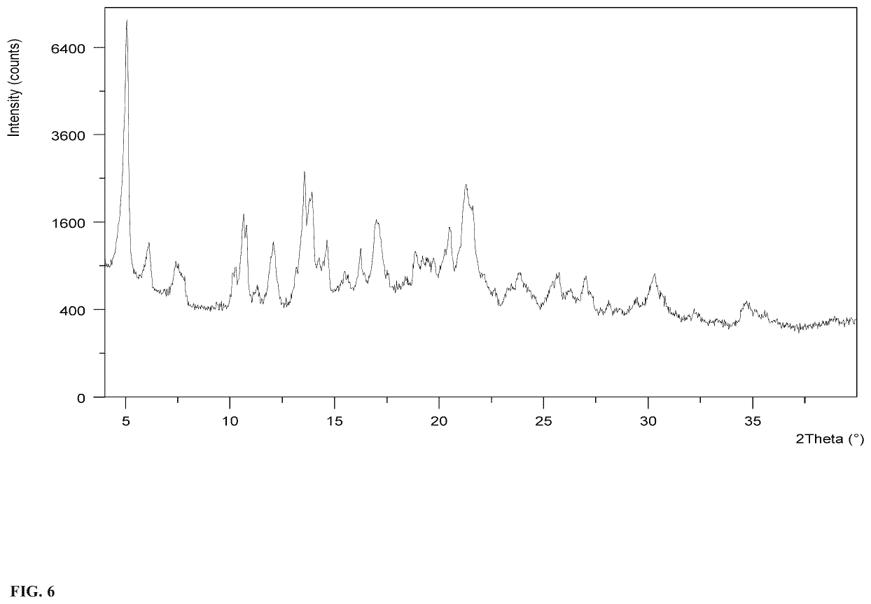

The solid was collected for XRPD analysis. The XRPD pattern of Form C of compound (I) is shown in FIG. 6. Major peaks and their related intensities in the XRPD pattern are shown in table below.

### Characterization Method:

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.026° at a scanning speed of 3.348°/min.

TABLE 6
X-ray powder diffraction peaks of Form C of compound (I)

| Pos. [°2-theta] | Rel. Int. [%] |
|---|---|
| 5.1 | 100 |
| 6.1 | 9 |
| 7.4 | 6 |
| 7.8 | 4 |
| 10.2 | 6 |
| 10.6 | 19 |
| 10.8 | 16 |
| 11.3 | 3 |
| 12.1 | 12 |
| 13.6 | 33 |
| 13.9 | 24 |
| 14.6 | 9 |
| 15.5 | 2 |
| 16.2 | 8 |

### Example 12: Preparation of Form E of Compound (I)

Approximate 10 mg of Form Amorphous of compound (I) as prepared in Example 7 was weighed and transferred to a centrifuge tube. The tube was placed inside a closed container filled with n-heptane, and let sit for 16 h.

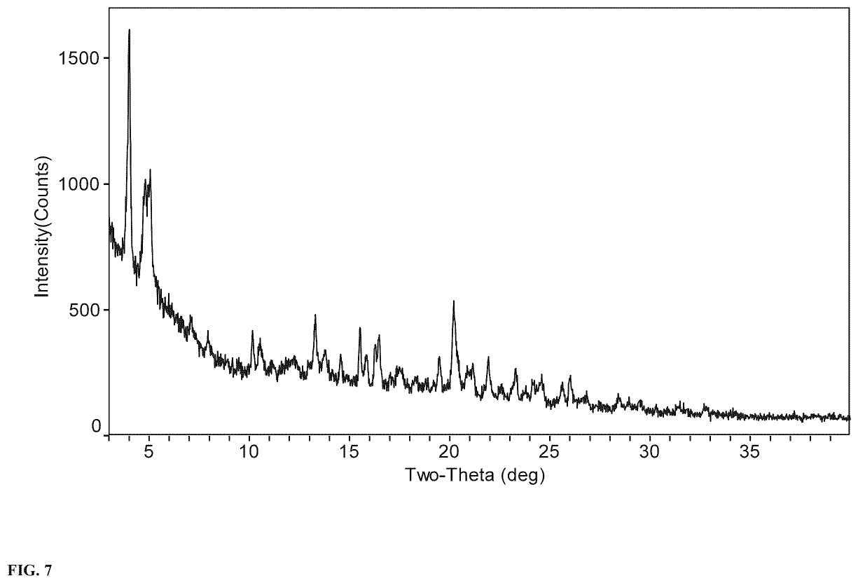

The solid was collected and analyzed by XRPD. The XRPD pattern of Form E of compound (I) is shown in FIG. 7. Major peaks and their related intensities in the XRPD pattern are shown in Table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 7
X-ray powder diffraction peaks of Form E of compound (I)

| Pos. [°2-theta] | Rel. Int. [%] |
|---|---|
| 4.0 | 100 |
| 5.1 | 65 |
| 5.4 | 39 |
| 10.2 | 26 |
| 10.5 | 24 |
| 11.8 | 19 |
| 12.2 | 20 |
| 12.9 | 18 |
| 13.3 | 30 |
| 13.8 | 21 |
| 14.6 | 20 |
| 15.5 | 26 |
| 15.8 | 20 |
| 16.5 | 25 |
| 17.0 | 15 |
| 17.5 | 17 |
| 19.5 | 19 |
| 20.2 | 33 |
| 21.2 | 18 |
| 21.9 | 19 |
| 22.6 | 13 |
| 23.3 | 16 |
| 23.8 | 12 |
| 24.1 | 14 |
| 24.6 | 15 |
| 25.6 | 13 |
| 26.0 | 15 |
| 26.8 | 11 |
| 28.4 | 10 |
| 29.0 | 9 |
| 29.5 | 9 |

### Example 13: Preparation of Form F of Compound (I)

Approximate 20 mg of Form Amorphous of compound (I) as prepared in Example 7 was weighed and transferred into a mortar. 0.1 mL n-propanol was added. The mixture was grinded manually for 3 minutes.

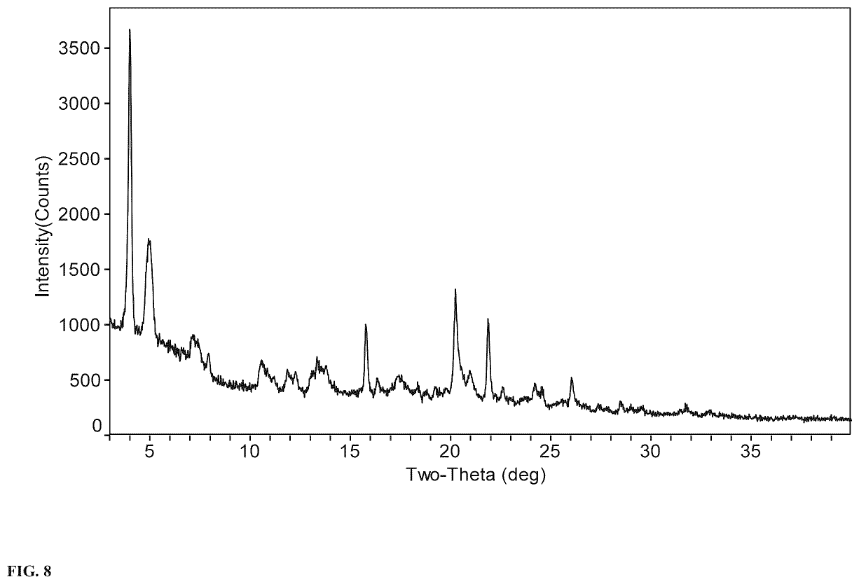

The solid was collected for XRPD analysis. The XRPD pattern of Form F of compound (I) is shown in FIG. 8. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 8
X-ray powder diffraction peaks of Form F of compound (I)

| Pos. [°2-theta] | Rel. Int. [%] |
|---|---|
| 4.0 | 100 |
| 4.9 | 48 |
| 7.1 | 25 |
| 7.4 | 24 |
| 7.9 | 20 |
| 10.6 | 18 |
| 11.2 | 14 |
| 11.9 | 16 |
| 12.3 | 15 |
| 13.1 | 16 |
| 13.3 | 19 |
| 13.8 | 17 |
| 15.8 | 27 |
| 16.3 | 14 |
| 17.4 | 15 |
| 17.9 | 12 |
| 18.4 | 13 |
| 19.2 | 12 |
| 19.8 | 11 |
| 20.3 | 36 |
| 21.0 | 16 |
| 21.9 | 29 |
| 22.6 | 12 |
| 23.8 | 9 |
| 24.2 | 13 |
| 24.6 | 12 |
| 25.6 | 9 |
| 26.0 | 14 |
| 28.5 | 8 |
| 29.0 | 7 |
| 29.6 | 7 |
| 31.7 | 8 |
| 32.9 | 6 |

### Example 14: Preparation of Form G of Compound (I)

Approximate 15 mg of Form A of compound (I) as prepared in Example 3 was weighed and transferred to a glass vial. 2 mL an ethanol/n-heptane mixture (1:1, v:v) was added and sonicated mildly to ensure complete dissolution. About 2 mg of PEG 6000 was added. The solution was evaporated to dryness at room temperature.

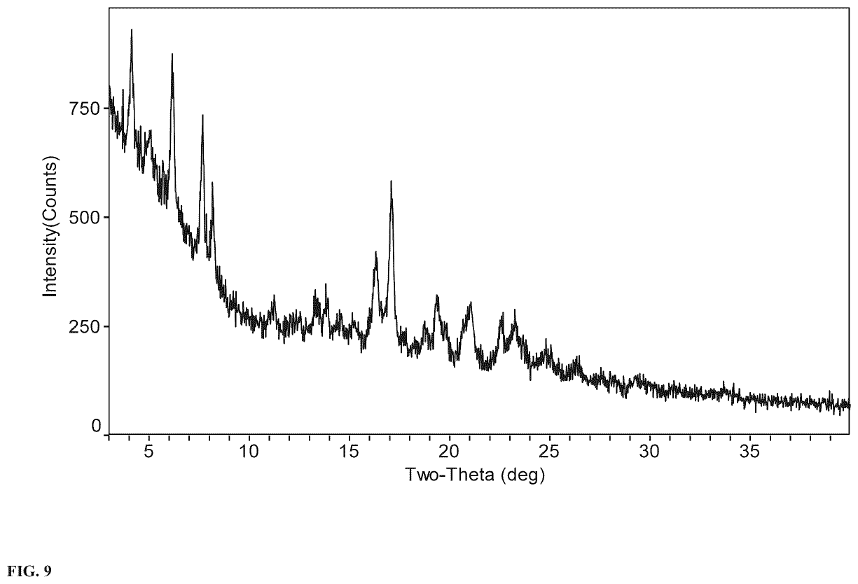

The solid was collected for XRPD analysis. The XRPD pattern of Form G of compound (I) is shown in FIG. 9. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 9
X-ray powder diffraction peaks of Form G of compound (I).

| Pos. [°2-theta] | Rel. Int. [%] |
|---|---|
| 3.7 | 85 |
| 4.1 | 100 |
| 5.0 | 75 |
| 6.2 | 94 |
| 7.7 | 79 |
| 8.2 | 62 |
| 11.3 | 34 |
| 13.3 | 36 |
| 13.8 | 37 |
| 14.5 | 31 |
| 16.3 | 45 |
| 17.1 | 63 |
| 18.8 | 28 |
| 19.3 | 34 |
| 19.9 | 27 |
| 20.8 | 30 |
| 21.1 | 33 |
| 22.7 | 30 |
| 23.3 | 31 |
| 23.8 | 23 |
| 24.8 | 24 |
| 25.1 | 22 |
| 26.5 | 19 |
| 29.4 | 15 |

### Example 15: Preparation of Sodium Salt Form J of Compound (I)

1.0 g of Form A of compound (I) as prepared in Example 3 was weighed into a vial and dissolved in 13 mL ethanol. The solution was stirred for 5 min under a 40° C. water bath. 73.19 mg of sodium hydroxide (1.1 eq.) was added into the solution and stirring was applied for 1 min. The solution became clear, then turned cloudy, and then became jell-like. 2.0 mL ethanol was added and the mixture was agitated at RT until the solution became flowable. After being agitated at RT for 5 h, the product was isolated by vacuum filtration. The wet cake was washed using a small amount of ethanol and dried at 40° C. in an air-blow oven for 16 h.

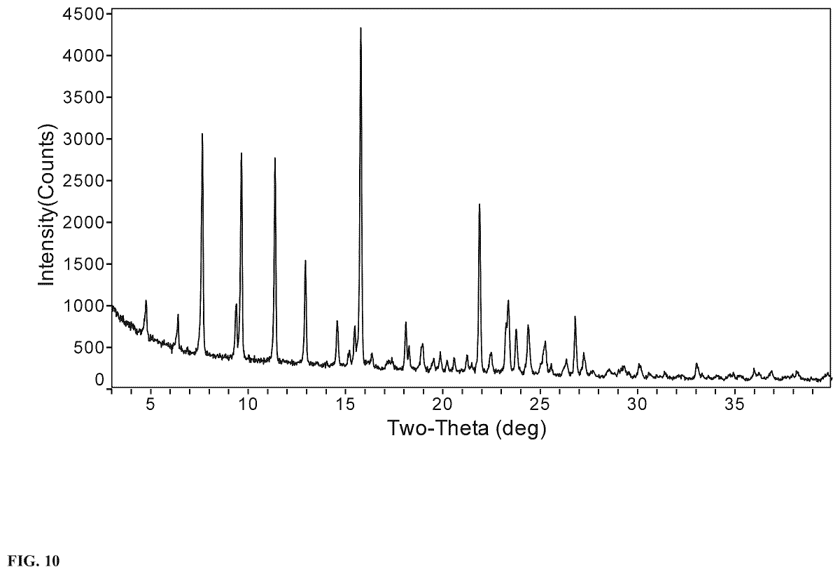

The solid was collected for XRPD analysis. The XRPD pattern of sodium salt Form J of compound (I) is shown in FIG. 10. Major peaks and their related intensities in the XRPD pattern are shown in table below.

### Experimental Conditions:

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.053° at a scanning speed of 10.504°/min.

TABLE 10
X-ray powder diffraction peaks of
sodium salt form J of compound (I).

| Pos. [°2-theta] | Rel. Int. [%] |
|---|---|
| 4.8 | 6 |
| 6.5 | 7 |
| 7.7 | 37 |
| 9.4 | 8 |
| 9.7 | 29 |
| 11.5 | 24 |
| 13.0 | 18 |
| 14.7 | 50 |
| 15.3 | 16 |
| 15.9 | 100 |
| 16.5 | 14 |
| 17.5 | 6 |
| 18.2 | 6 |
| 19.0 | 17 |
| 19.6 | 5 |
| 20.0 | 10 |
| 20.3 | 7 |
| 20.7 | 7 |
| 21.3 | 10 |
| 22.0 | 32 |
| 22.6 | 11 |
| 23.4 | 28 |
| 23.9 | 24 |
| 24.5 | 20 |
| 25.3 | 14 |
| 26.4 | 7 |
| 26.9 | 9 |
| 27.3 | 6 |
| 27.7 | 2 |
| 28.6 | 2 |
| 29.4 | 5 |
| 30.2 | 6 |
| 30.7 | 1 |
| 31.5 | 2 |
| 32.3 | 1 |
| 33.2 | 3 |
| 34.2 | 2 |
| 34.7 | 1 |
| 35.5 | 1 |
| 36.1 | 3 |
| 37.0 | 3 |
| 38.3 | 3 |
| 39.1 | 1 |

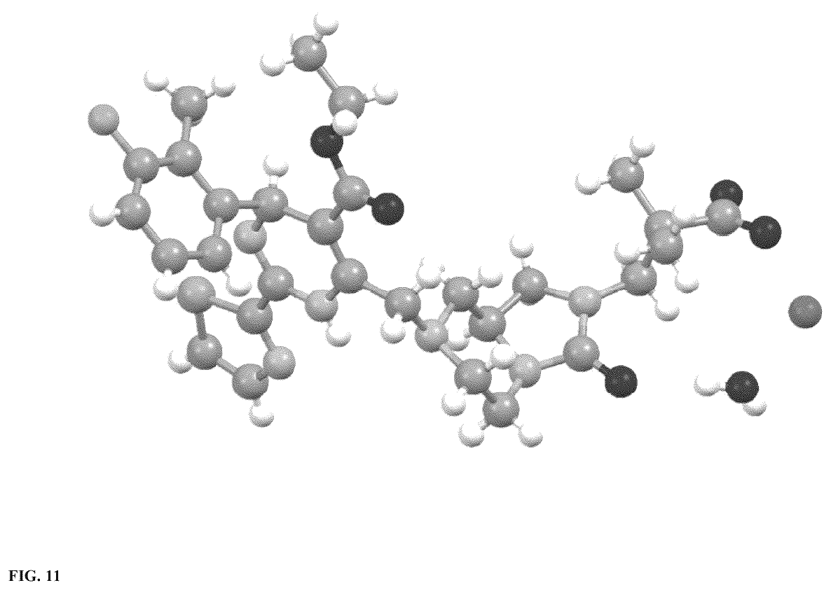

FIG. 11 shows the X-ray structure of sodium salt Form J. The single crystal X-ray intensity data were collected at 293(2) K using a Bruker SMART APEX II with Mo-K-alpha-radiation (0.71 Å). Structure solution and refinement was performed using the She1XTL software (Bruker AXS, Karlsruhe). The crystal data and structure refinement is shown in Table 11.

TABLE 11
Single crystal structural data of sodium salt Form J

| Crystal form | Sodium salt Form J | |
|---|---|---|
| Solid form description | Hydrate | |
| Measuring Temperature | 293(2)K | |
| Crystal system | orthorhombic | |
| Space group | P 2₁2₁2₁ | |
| Unit cell dimensions | 6.0795(6) | Å |
| a= | | |
| b= | 14.5770(13) | Å |
| c= | 36.065(3) | Å |
| α= | 90° | |
| β= | 90° | |
| γ= | 90° | |
| Cell volume | 3196.1(5) | Å³ |
| API molecules in unit cell | 4 | |
| Calculated density | 1.327 | g/cm³ |

### Example 16: Preparation of Form H of Compound (I)

200 mg of sodium salt Form J of compound (I) as prepared in Example 15 was weighed into a vial, to which 20 mL FaSSIF solution was added to form a suspension. The obtained suspension was agitated at 25° C. for 16 h. Then, the solid was collected by filtration and dried at 40° C. under air blowing for 16 h. The solid was collected for XRPD analysis, DSC analysis and TGA analysis.

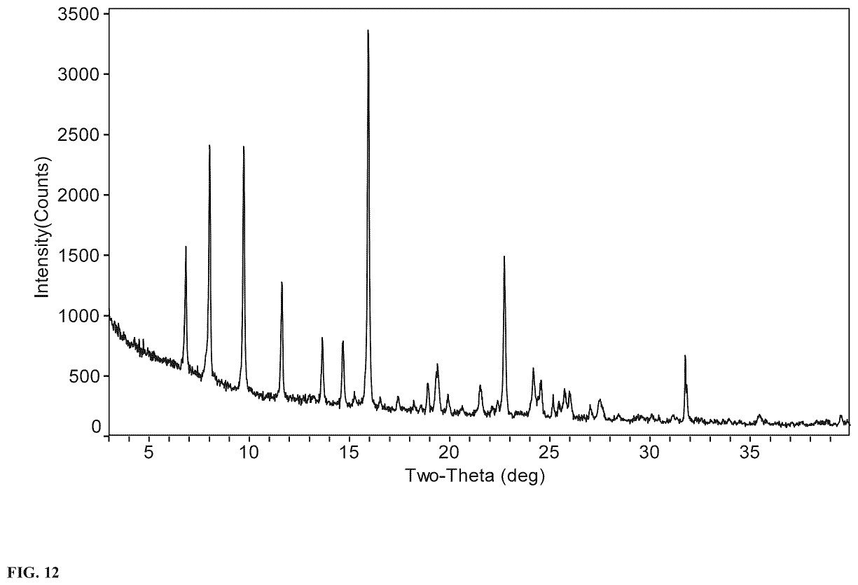

The XRPD pattern of Form H of compound (I) is shown in FIG. 12. Major peaks and their related intensities in the XRPD pattern are shown in table below.

### Characterization Method

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.026° at a scanning speed of 3.348°/min.

DSC analysis: TA Q2000, 25-250° C., heating rate 10° C./min.

TGA analysis: TA Q5000, 25-300° C., heating rate 10° C./min.

TABLE 12
X-ray powder diffraction peaks of Form H of compound (I).

| Pos. [°2-theta] | Rel. Int. [%] |
|---|---|
| 6.8 | 27 |
| 8.0 | 46 |
| 9.7 | 34 |
| 11.6 | 20 |
| 13.6 | 11 |
| 14.6 | 67 |
| 15.2 | 19 |
| 15.7 | 33 |
| 15.9 | 100 |
| 16.5 | 11 |
| 17.4 | 14 |
| 18.2 | 1 |
| 18.5 | 5 |
| 18.9 | 29 |
| 19.3 | 14 |
| 19.4 | 8 |
| 19.9 | 17 |
| 20.6 | 6 |
| 21.5 | 14 |
| 22.3 | 9 |
| 22.7 | 28 |
| 23.5 | 1 |
| 24.1 | 39 |
| 24.5 | 29 |
| 25.1 | 13 |
| 25.7 | 8 |
| 26.0 | 18 |
| 26.3 | 1 |
| 27.0 | 8 |
| 27.5 | 10 |
| 28.4 | 2 |
| 29.1 | 2 |
| 29.5 | 6 |
| 29.9 | 3 |
| 30.2 | 4 |
| 30.4 | 1 |
| 31.1 | 6 |
| 31.4 | 3 |
| 32.0 | 2 |
| 32.2 | 2 |
| 32.7 | 1 |
| 33.0 | 1 |
| 33.3 | 1 |
| 33.9 | 1 |
| 34.2 | 1 |
| 34.5 | 1 |
| 35.3 | 3 |
| 35.8 | 3 |
| 36.4 | 1 |
| 37.5 | 1 |

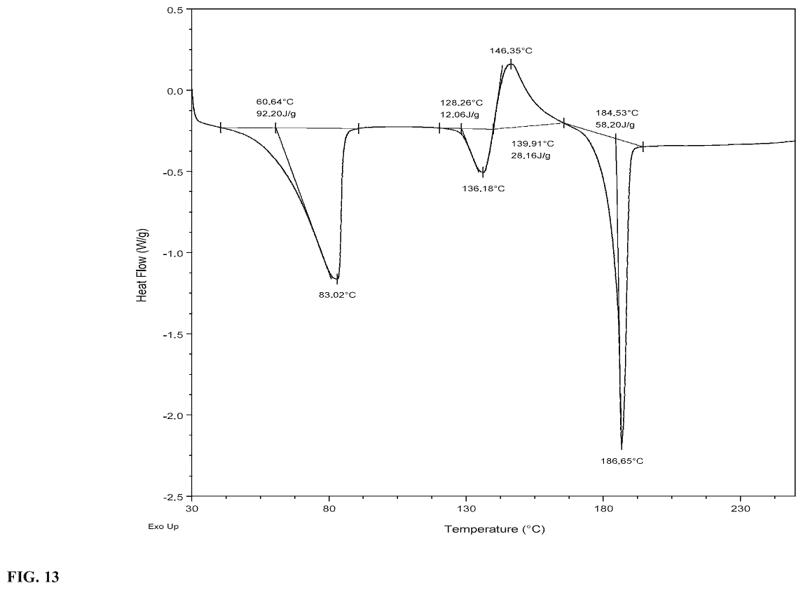

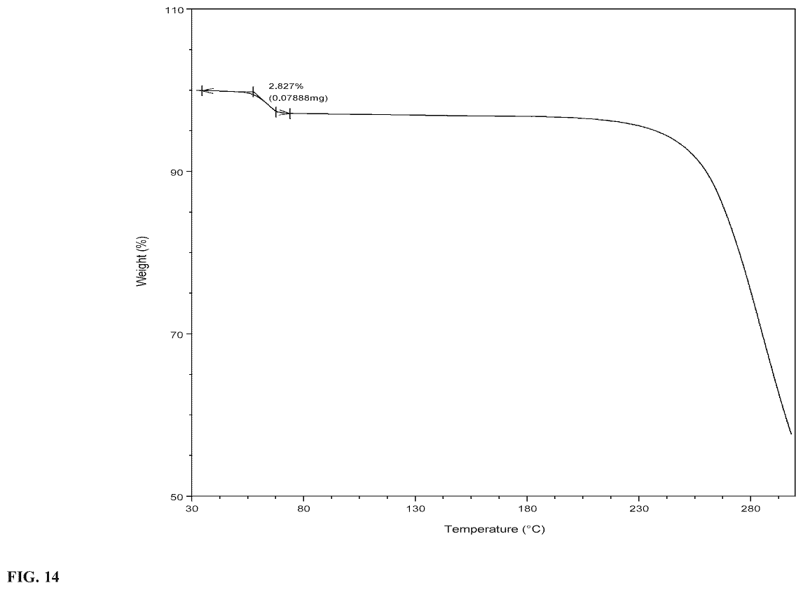

DSC and TGA results shown in FIG. 13. and FIG. 14. indicate Form H of compound (I) has a dehydration temperature at around 60° C.

DSC and TGA results shown in FIG. 13. and FIG. 14. indicate Form H of compound (I) has a dehydration temperature at around 60° C.

### Example 17: Preparation of Form I of Compound (I)

10 mg of Form H of compound (I) as prepared in Example 16 was weighed into a variable temperature chamber. The sample was placed at 60° C. for 5 min.

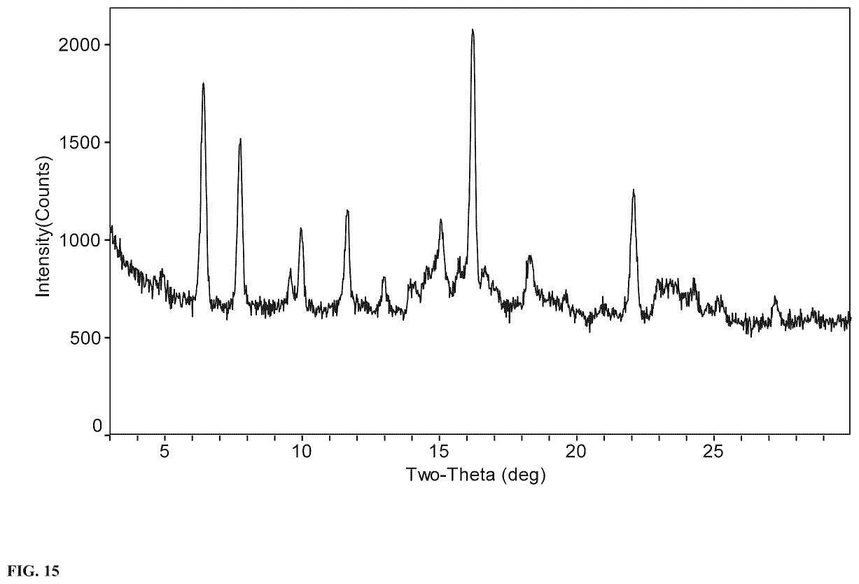

The solid was collected for XRPD analysis. The XRPD pattern of Form I of compound (I) is shown in FIG. 15. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 30 degree 2-theta. The step size was 0.02° at a scanning speed of 6°/min.

TABLE 13
X-ray powder diffraction peaks of Form I of compound (I).

| Pos. [°2-theta] | Rel. Int. [%] |
|---|---|
| 6.4 | 84 |
| 7.8 | 65 |
| 9.6 | 16 |
| 9.9 | 31 |
| 11.6 | 39 |
| 13.0 | 13 |
| 13.9 | 10 |
| 14.5 | 13 |
| 15.0 | 27 |
| 15.7 | 11 |
| 16.2 | 100 |
| 16.7 | 8 |
| 18.3 | 19 |
| 19.6 | 6 |
| 20.9 | 8 |
| 22.1 | 48 |
| 23.0 | 12 |
| 24.3 | 11 |
| 25.3 | 9 |
| 27.2 | 11 |

### Example 18: Preparation of Hydrochloride Salt Form K of compound (I)

400 mg of Form A of compound (I) as prepared in Example 3 and 9.0 mL acetone was added into a vial in a 45° C. water bath, and the mixture was stirred to afford a clear solution. 74.4 mg concentrated hydrochloric acid (1.1 eq.) in 1.0 mL acetone was added to the solution and the solution instantly became cloudy. After being agitated at RT for 1 h, the mixture became sticky and solidified. After addition of 2.0 mL acetone, the mixture became flowable. The suspension was agitated for another 5 h at RT, the solid was collected by vacuum filtration, washed with a small amount of acetone, and dried at 40° C. in an air-blow oven for 16 h. The solid was collected for XRPD analysis.

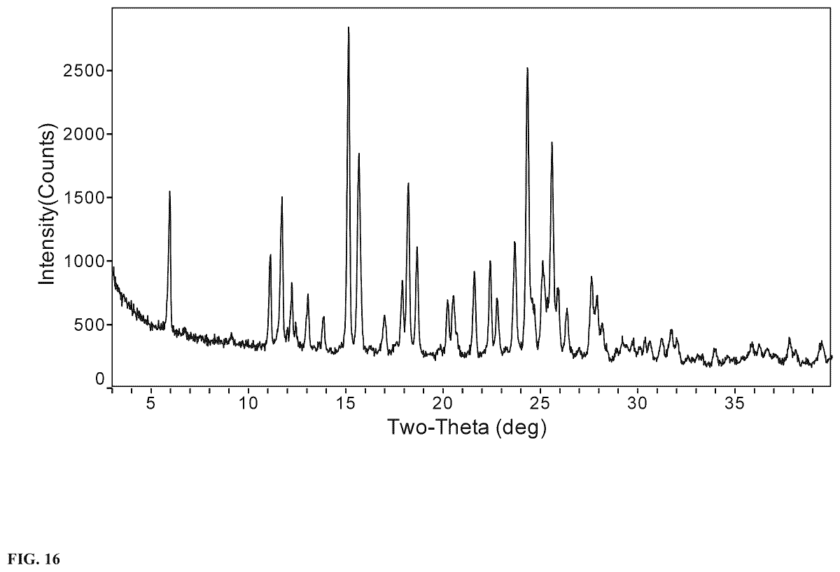

The XRPD pattern of hydrochloride salt Form K of compound (I) is shown in FIG. 16. Major peaks and their related intensities in the XRPD pattern are shown in table below.

### Experimental Conditions

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.026° at a scanning speed of 3.348°/min.

TABLE 14
X-ray powder diffraction peaks of hydrochloride
salt Form K of compound(I).

| Pos. [°2-theta] | Rel. Int. [%] |
|---|---|
| 5.4 | 100 |
| 5.8 | 6 |
| 6.9 | 8 |
| 7.9 | 1 |
| 9.6 | 9 |
| 10.9 | 3 |
| 11.7 | 3 |
| 12.0 | 1 |
| 12.5 | 9 |
| 13.3 | 46 |
| 13.8 | 25 |
| 14.8 | 32 |
| 15.9 | 48 |
| 16.3 | 55 |
| 16.5 | 14 |
| 18.0 | 48 |
| 18.8 | 1 |
| 18.9 | 1 |
| 19.3 | 9 |
| 19.5 | 19 |
| 20.0 | 15 |
| 20.6 | 2 |
| 20.9 | 6 |
| 21.4 | 2 |
| 21.7 | 24 |
| 22.1 | 1 |
| 22.4 | 15 |
| 22.7 | 40 |
| 23.4 | 26 |
| 23.6 | 7 |
| 23.8 | 9 |
| 24.1 | 15 |
| 24.7 | 2 |
| 25.4 | 9 |
| 25.6 | 6 |
| 26.1 | 4 |
| 26.7 | 5 |
| 27.3 | 1 |
| 27.8 | 10 |
| 28.0 | 15 |
| 29.6 | 7 |
| 30.3 | 2 |
| 31.3 | 1 |
| 31.8 | 2 |
| 32.9 | 3 |
| 34.1 | 2 |
| 34.8 | 4 |
| 35.3 | 2 |
| 35.7 | 2 |
| 35.9 | 2 |

### Example 19: Preparation of Hydrochloride Salt Form L of Compound (I)

150 mg of hydrochloride salt Form K of compound (I) as prepared in Example 18 was placed in a high relative humidity chamber closed to 100% RH at ambient temperature for 3 days.

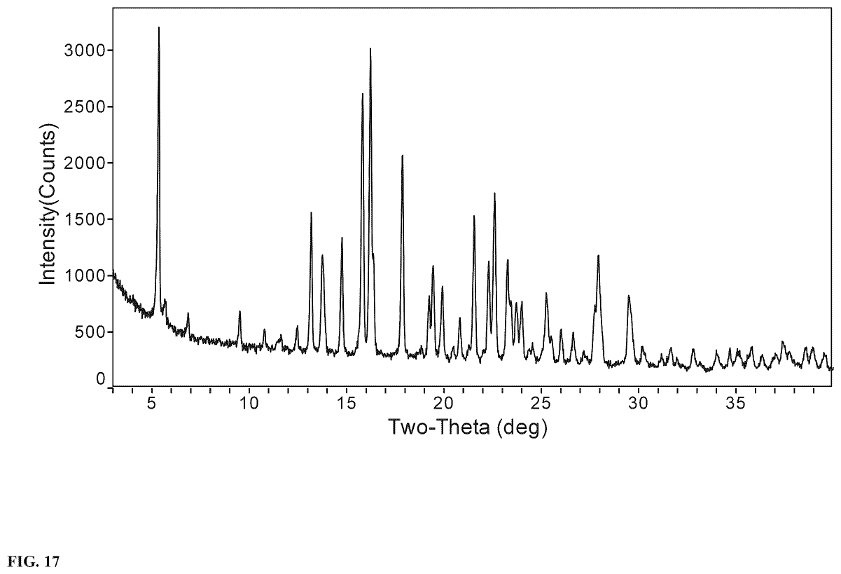

The solid was collected for XRPD analysis. The XRPD pattern of hydrochloride salt Form L of compound (I) is shown in FIG. 17. Major peaks and their related intensities in the XRPD pattern are shown in table below.

### Experimental Method

XRPD: PANalytical EMPYREAN X-ray powder diffractometer with Cu-Kα radiation. Tube voltage was 40 KV and tube current was 40 mA. Scan range was from 4 to 40 degree 2-theta. The step size was 0.026° at a scanning speed of 3.348°/min.

TABLE 15
X-ray powder diffraction peaks of hydrochloride
salt Form L of compound(I).

| Pos. [°2-theta] | Rel. Int. [%] |
|---|---|
| 6.0 | 94 |
| 11.2 | 45 |
| 11.5 | 1 |
| 11.8 | 56 |
| 12.1 | 7 |
| 12.3 | 22 |
| 12.5 | 8 |
| 13.1 | 24 |
| 14.0 | 10 |
| 15.3 | 100 |
| 15.8 | 58 |
| 17.1 | 12 |
| 17.7 | 3 |
| 18.0 | 16 |
| 18.3 | 56 |
| 18.7 | 29 |
| 20.4 | 15 |
| 20.6 | 14 |
| 21.7 | 22 |
| 22.5 | 29 |
| 22.9 | 14 |
| 23.8 | 31 |
| 24.4 | 66 |
| 24.8 | 10 |
| 25.2 | 19 |
| 25.7 | 41 |
| 26.0 | 14 |
| 26.5 | 9 |
| 27.1 | 1 |
| 27.7 | 21 |
| 28.0 | 11 |
| 28.3 | 7 |
| 29.3 | 4 |
| 29.8 | 4 |
| 30.5 | 4 |
| 30.8 | 4 |
| 31.3 | 5 |
| 31.8 | 6 |
| 32.1 | 6 |
| 32.7 | 1 |
| 33.1 | 2 |
| 34.0 | 4 |
| 34.7 | 2 |
| 35.9 | 4 |
| 36.3 | 4 |
| 36.8 | 4 |
| 37.2 | 2 |
| 37.8 | 7 |
| 38.2 | 3 |
| 38.8 | 1 |

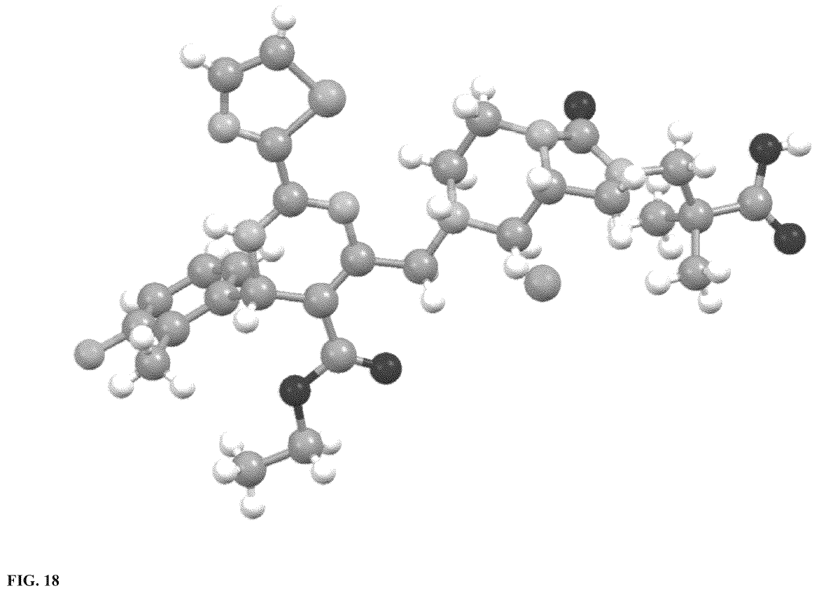

FIG. 18 shows the X-ray structure of hydrochloride salt Form L. The single crystal X-ray intensity data were collected at 100.08 K using a Gemini with Mo-K-alpha-radiation (0.71 Å). Structure solution and refinement was performed using the Olex2 software. The crystal data and structure refinement is shown in Table 16.

TABLE 16
Single crystal structural data of hydrochloride salt Form L

| Crystal form | Hydrochloride salt Form L | |
|---|---|---|
| Solid form description | Hydrate | |
| Measuring Temperature | 100.08K | |
| Crystal system | triclinic | |
| Space group | P 1 | |
| Unit cell dimensions | 10.0621(3) | Å |
| a= | | |
| b= | 11.9420(5) | Å |
| c= | 15.6269(5) | Å |
| α= | 103.562(3)° | |
| β= | 105.711(3)° | |
| γ= | 93.148(3)° | |
| Cell volume | 1743.06(11) | Å³ |
| API molecules in unit cell | 2 | |
| Calculated density | 1.356 | g/cm³ |

### Example 20: Preparation of Sulfate Salt Form M of Compound (I)

9.98 mg of Form A of compound (I) as prepared in Example 3 was added into 1.5 mL IPA, and 1.8 mg of sulfuric acid (1.1 eq.) was added to obtain a clear solution. The solvent was evaporated to 0.2 mL and remaining was agitated for another 16 h, resulting in a suspension. The solid was collected by centrifugation and dried at 40° C. in a vacuum oven for 16 h.

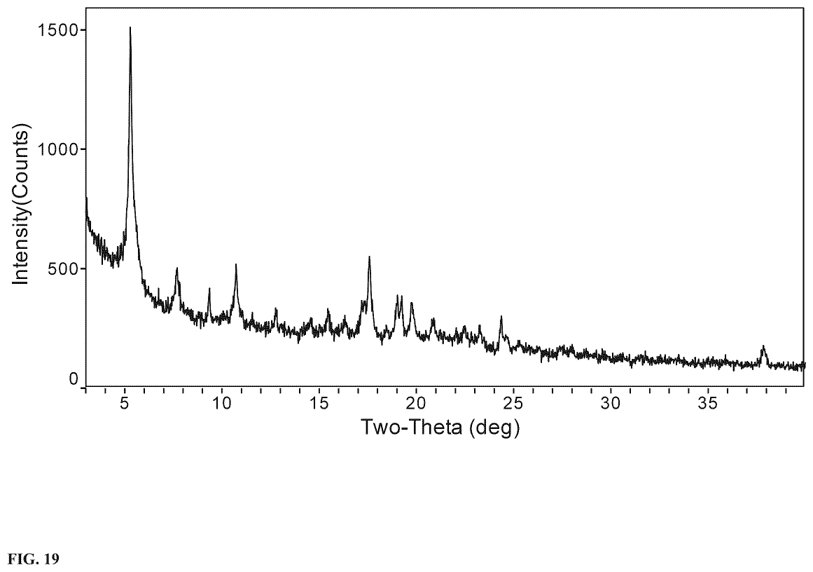

The solid was collected for XRPD analysis. The XRPD pattern of sulfate Form M of compound (I) is shown in FIG. 19. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 17
X-ray powder diffraction peaks of
sulfate Form M of compound (I).

| Pos. [°2-theta] | Rel. Int. [%] |
|---|---|
| 5.3 | 100 |
| 7.7 | 18 |
| 9.4 | 12 |
| 10.7 | 23 |
| 12.8 | 9 |
| 14.6 | 7 |
| 15.5 | 10 |
| 16.3 | 7 |
| 17.2 | 14 |
| 17.6 | 32 |
| 19.0 | 17 |
| 19.2 | 17 |
| 19.8 | 13 |
| 20.9 | 8 |
| 22.1 | 5 |
| 22.5 | 6 |
| 23.3 | 8 |
| 24.4 | 14 |
| 24.7 | 5 |
| 29.6 | 4 |
| 35.9 | 4 |
| 37.8 | 8 |

### Example 21: Preparation of Sulfate Salt Form N of Compound (I)

401 mg of Form A of compound (I) as prepared in Example 3 and 8.0 mL IPA were added into a vial and heated to 60° C. in a water bath. The solution became clear after agitation, then was cooled to RT and became slightly cloudy. 76.6 mg of sulfuric acid (about 1.1 eq.) diluted in 1.0 mL IPA was added, resulting in a clear solution. The solution was agitated for 0.5 h at RT and then for 16 h at 10° C., no precipitation occurred. The solvent was evaporated to 2-3 mL, which was agitated at 10° C. The solution became very cloudy within 2 min and continuous agitation at 10° C. resulted in suspension (which turned to oil after exposure to air). After 5.0 mL IPE was added drop-wise at 10° C., the mixture was heated to RT and agitated for 16 h. The solid was isolated by vacuum filtration, and air-dried at RT. 200 mg of resulted solid was added into 1.0 mL EtOAc, which was agitated for 24 h at RT. Solid was collected by filtration, washed with a small amount of EtOAc, and dried at 40° C. in an air-blow oven for 24 hours.

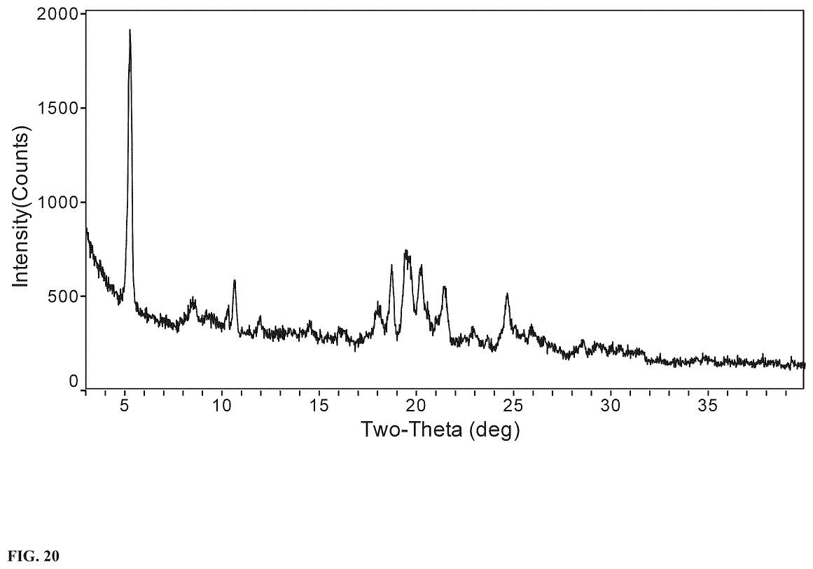

The solid was collected for XRPD analysis. The XRPD pattern of sulfate Form N of compound (I) is shown in FIG. 20. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 18
X-ray powder diffraction peaks of sulfate Form N of compound (I).

| Pos. [°2-theta] | Rel. Int. [%] |
|---|---|
| 5.3 | 100 |
| 8.5 | 10 |
| 9.2 | 5 |
| 10.3 | 9 |
| 10.7 | 19 |
| 12.0 | 7 |
| 14.5 | 6 |
| 18.0 | 13 |
| 18.7 | 24 |
| 19.4 | 27 |
| 20.3 | 19 |
| 21.0 | 5 |
| 21.5 | 18 |
| 22.9 | 7 |
| 23.7 | 4 |
| 24.7 | 19 |
| 25.1 | 6 |
| 25.9 | 6 |
| 28.6 | 5 |
| 29.2 | 4 |
| 29.3 | 3 |
| 30.1 | 4 |
| 36.0 | 3 |
| 37.8 | 4 |

### Example 22: Preparation of Besylate Salt Form O of Compound (I)

401 mg of Form A of compound (I) as prepared in Example 3 and 12.0 mL ethyl acetate was added into a vial at 65° C., and agitated until the solution became clear. The solution was cooled to RT and it turned slightly cloudy. 122.64 mg benzensulfonic acid (1.2 eq.) in 0.5 mL IPA was added to the solution, which turned clear again. The solution was stirred for 0.5 hour at RT then 16 hours at 10° C., precipitations occurred. The suspension was kept stirring at RT for 3 days. Creamy solids were collected by filtration, and were dried at RT.

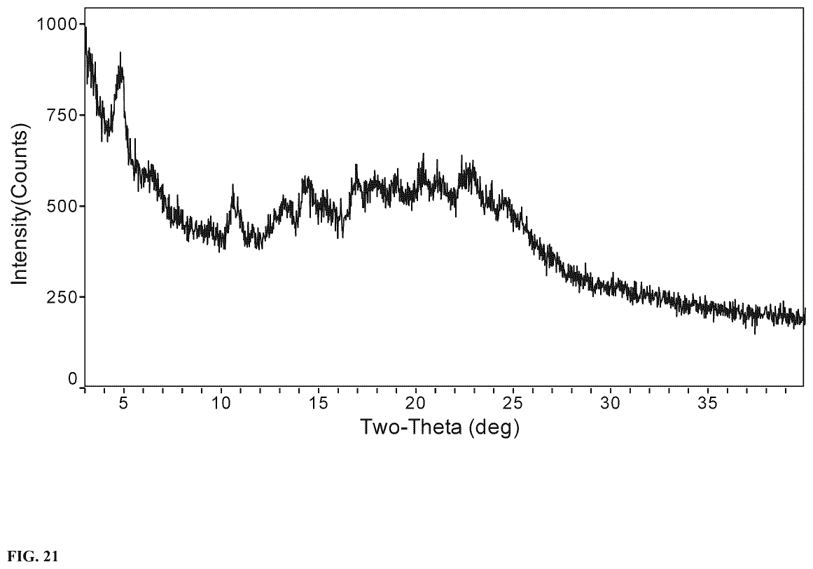

The solid was collected for XRPD analysis. The XRPD pattern of besylate Form O of compound (I) is shown in FIG. 21. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 19
X-ray powder diffraction peaks of
besylate Form O of compound (I).

| Pos. [°2-theta] | Rel. Int. [%] |
|---|---|
| 4.9 | 100 |
| 10.6 | 68 |
| 13.2 | 41 |
| 14.3 | 51 |
| 16.9 | 48 |
| 17.9 | 21 |
| 19.1 | 33 |
| 20.2 | 31 |
| 21.1 | 40 |
| 22.4 | 54 |
| 22.9 | 55 |
| 23.9 | 35 |
| 24.4 | 32 |

### Example 23: Preparation of Potassium Salt Form P of Compound (I)

10.02 mg of Form A of compound (I) as prepared in Example 3 was dissolved in 0.3 mL MeOH. 1.22 mg of potassium hydroxide (1.1 eq.) was added into the solution, which was agitated to obtain a clear solution. After being agitated for another 16 hours, the solvent was then reduced to 0.2 mL, and agitation continued at 10° C. for another 16hours. 3.0 mL n-heptane was added to the solution, then a small amount of solid precipitated. The solid was collected by centrifuge, and dried at 40° C. under vacuum for 24 hours.

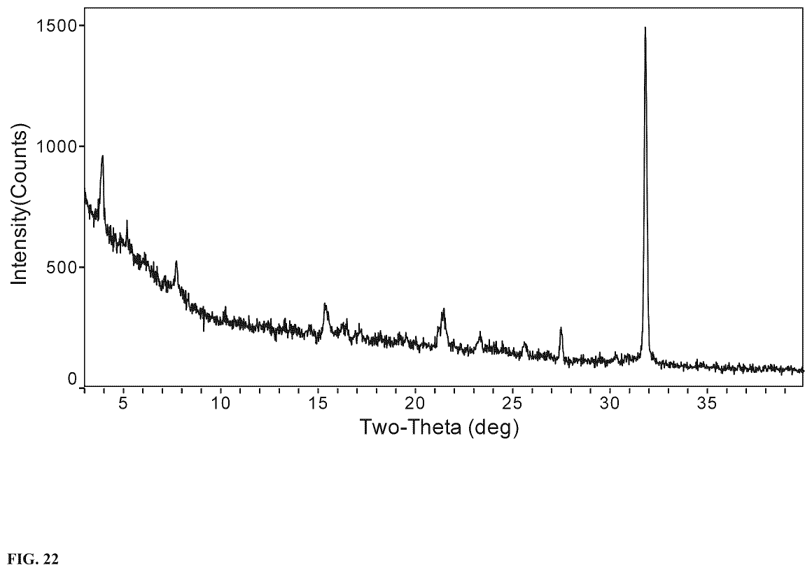

The solid was collected for XRPD analysis. The XRPD pattern of potassium salt Form P of compound (I) is shown in FIG. 22. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 20
X-ray powder diffraction peaks of potassium
salt Form P of compound (I).

| Pos. [°2-theta] | Rel. Int. [%] |
|---|---|
| 3.9 | 21 |
| 5.2 | 9 |
| 7.7 | 10 |
| 10.2 | 5 |
| 13.3 | 5 |
| 13.5 | 4 |
| 15.3 | 10 |
| 16.3 | 4 |
| 16.5 | 6 |
| 17.1 | 5 |
| 19.5 | 4 |
| 21.2 | 7 |
| 21.5 | 12 |
| 23.3 | 5 |
| 23.8 | 4 |
| 25.6 | 4 |
| 27.5 | 10 |
| 29.5 | 3 |
| 30.3 | 3 |
| 31.8 | 100 |

### Example 24: Preparation of Potassium Salt Form Q of Compound (I)

705.70 mg of Form A of compound (I) as prepared in Example 1 was dissolved in 50 mL ethyl acetate. The solution in a vial was placed in a 40° C. water bath and agitated to ensure complete dissolution, then 218.28 mg of potassium phthalimide (1.0 eq.) was added and the solution turned slightly cloudy. The solution was stirred for 16 hours at RT which became significantly cloudy. The solid was collected by filtration and washed by 10 mL ethyl acetate, and dried at 40° C. in an air-blow oven for 5 hours.

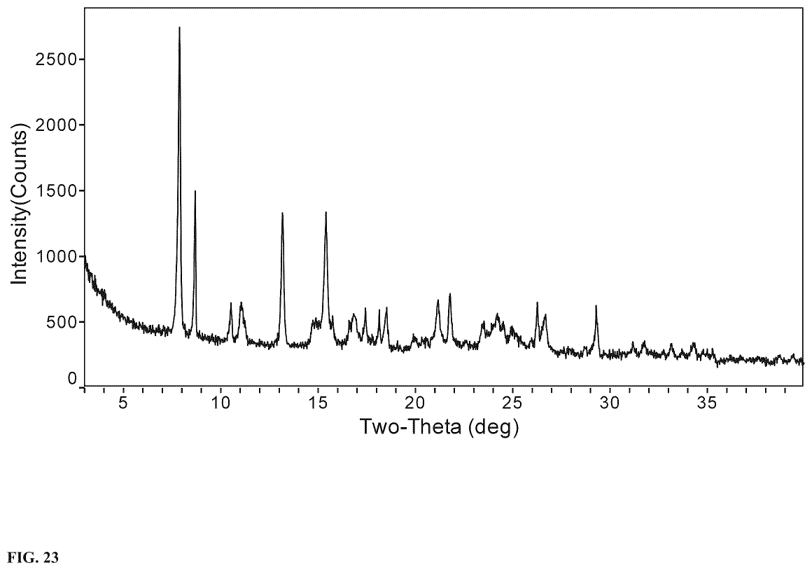

The solid was collected for XRPD analysis. The XRPD pattern of potassium salt Form Q of compound (I) is shown in FIG. 23. Major peaks and their related intensities in the XRPD pattern are shown in table below.

### Experimental Conditions

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 21
X-ray powder diffraction peaks of potassium
salt Form Q of compound (I).

| Pos. [°2-theta] | Rel. Int. [%] |
|---|---|
| 7.9 | 100 |
| 8.7 | 47 |
| 10.5 | 12 |
| 11.0 | 13 |
| 13.2 | 43 |
| 14.7 | 4 |
| 14.9 | 8 |
| 15.4 | 43 |
| 15.7 | 7 |
| 16.8 | 10 |
| 17.4 | 11 |
| 18.1 | 11 |
| 18.5 | 12 |
| 19.9 | 4 |
| 21.2 | 14 |
| 21.8 | 16 |
| 23.9 | 6 |
| 24.2 | 9 |
| 24.5 | 5 |
| 25.0 | 5 |
| 26.0 | 3 |
| 26.3 | 16 |
| 26.7 | 12 |
| 27.6 | 2 |
| 27.9 | 2 |
| 28.7 | 3 |
| 29.3 | 16 |
| 31.2 | 4 |
| 31.8 | 5 |
| 33.2 | 4 |
| 33.7 | 2 |
| 34.3 | 5 |
| 34.8 | 3 |
| 35.3 | 4 |
| 38.7 | 2 |
| 39.4 | 3 |

### Example 25: Preparation of Potassium Salt Form R of Compound (I)

5.0 mg of potassium salt Form Q of compound (I) as prepared in Example 24 was suspeneded in 0.5 mL IPAc. The suspension was stirred at RT for 3 days.

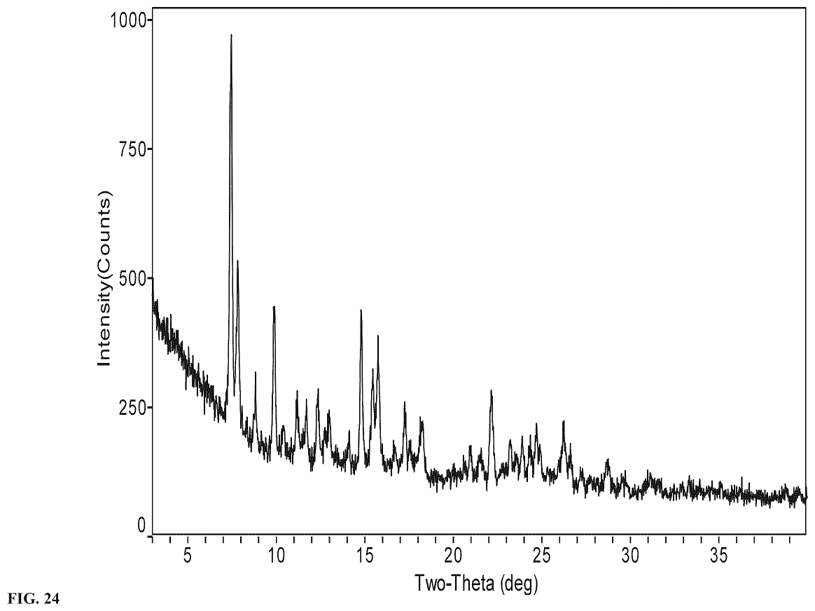

The solid was collected for XRPD analysis. The XRPD pattern of potassium salt Form R of compound (I) is shown in FIG. 24. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 22
X-ray powder diffraction peaks of potassium
salt Form R of compound (I).

| Pos. [°2-theta] | Rel. Int. [%] |
|---|---|
| 7.5 | 100 |
| 7.8 | 42 |
| 8.8 | 18 |
| 9.9 | 37 |
| 10.4 | 7 |
| 11.2 | 16 |
| 11.7 | 15 |
| 12.4 | 18 |
| 13.0 | 13 |
| 14.1 | 8 |
| 14.8 | 39 |
| 15.4 | 25 |
| 15.7 | 33 |
| 17.2 | 16 |
| 17.6 | 8 |
| 18.3 | 12 |
| 20.6 | 5 |
| 21.0 | 8 |
| 21.6 | 7 |
| 22.2 | 23 |
| 23.2 | 9 |
| 23.9 | 8 |
| 24.3 | 9 |
| 24.7 | 12 |
| 26.3 | 15 |
| 26.6 | 10 |
| 27.3 | 5 |
| 28.8 | 8 |
| 31.1 | 5 |
| 36.3 | 5 |

### Example 26: Preparation of Potassium Salt Form S of Compound (I)

10 mg of potassium salt Form Q of compound (I) as prepared in Example 24 was weighed into a variable temperature chamber. The sample was placed at 120° C. for 5 minutes.

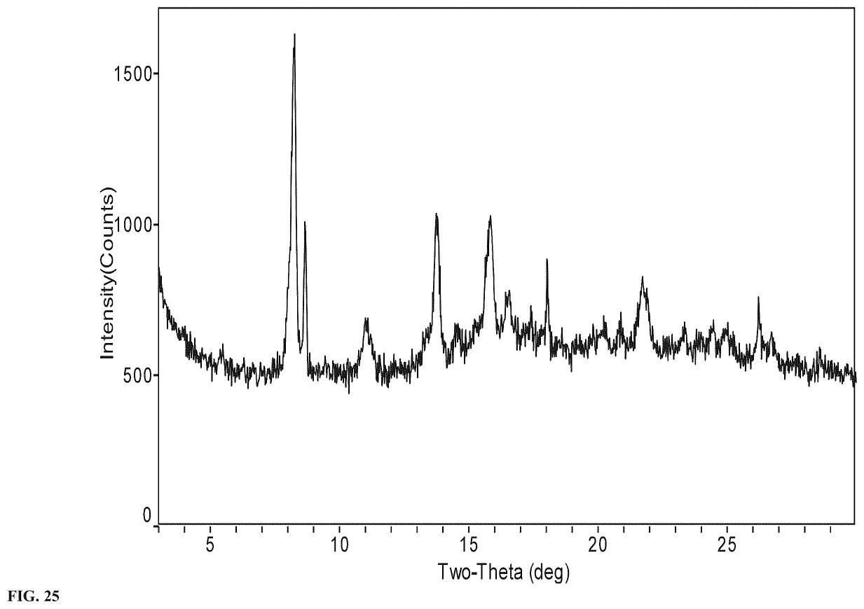

The solid was collected for XRPD analysis. The XRPD pattern of potassium salt Form S of compound (I) is shown in FIG. 25. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 30 degree 2-theta. The step size was 0.02° at a scanning speed of 6°/min.

TABLE 23
X-ray powder diffraction peaks of potassium
salt Form S of compound (I).

| Pos. [°2-theta] | Rel. Int. [%] |
|---|---|
| 5.5 | 7 |
| 8.3 | 100 |
| 8.7 | 44 |
| 11.0 | 17 |
| 11.2 | 11 |
| 12.1 | 7 |
| 13.4 | 11 |
| 13.7 | 44 |
| 14.6 | 8 |
| 15.8 | 37 |
| 16.6 | 12 |
| 18.0 | 25 |
| 19.8 | 7 |
| 20.3 | 7 |
| 20.9 | 9 |
| 21.7 | 20 |
| 24.5 | 9 |
| 24.9 | 7 |
| 26.2 | 19 |
| 26.7 | 9 |
| 28.6 | 9 |

### Example 27: Preparation of Calcium Salt Form T of Compound (I)

20.04 mg of Sodium salt Form J of compound (I) as prepared in Example 15 was dissolved in 0.7 mL water at RT, to which was added 4.10 mg anhydrous calcium chloride (1.1 eq.) in 0.1 mL water, and an emulsion like white suspension formed. Additional 0.4 mL water was added and the suspension was agitated at RT for 1.5 hours. The solid was collected by centrifugation and dried for 16 hours at 40° C. in a vacuum oven.

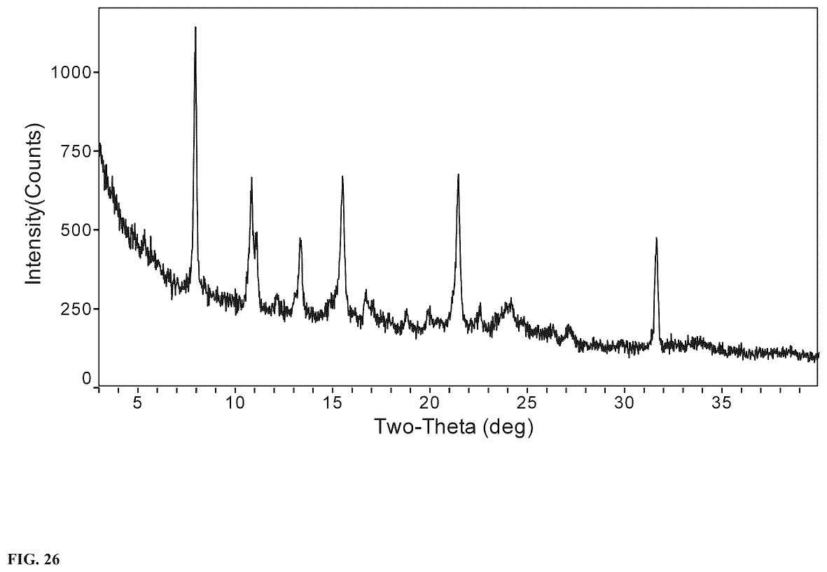

The solid was collected for XRPD analysis. The XRPD pattern of calcium salt Form T of compound (I) is shown in FIG. 26. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 24
X-ray powder diffraction peaks of calcium
salt Form T of compound (I).

| Pos. [°2-theta] | Rel. Int. [%] |
|---|---|
| 5.3 | 10 |
| 8.0 | 100 |
| 10.8 | 48 |
| 11.1 | 29 |
| 13.3 | 29 |
| 14.9 | 9 |
| 15.5 | 52 |
| 16.7 | 12 |
| 17.1 | 8 |
| 18.8 | 8 |
| 20.0 | 8 |
| 21.5 | 57 |
| 22.6 | 9 |
| 23.9 | 9 |
| 24.2 | 12 |
| 27.1 | 7 |
| 31.6 | 41 |
| 36.0 | 4 |

### Example 28: Preparation of Calcium Salt Form U of Compound (I)

304.32 mg of sodium salt Form J of compound (I) as prepared in Example 15 was dissolved in 10 mL water at RT with sonication. About 59.90 mg of anhydrous calcium chloride (1.1 eq.) in 1.0 mL water was added dropwise to the above solution, the solution instantly became cloudy. After 1 hour of agitation at RT, the suspension became sticky and solidified. It became flowable after addition of 4.0 mL water with agitation for 16 hours at RT, the solid was collected by filtration under vacuum, washed with a small amount of water and dried at 40° C. in an air-blow oven for 16 hours.

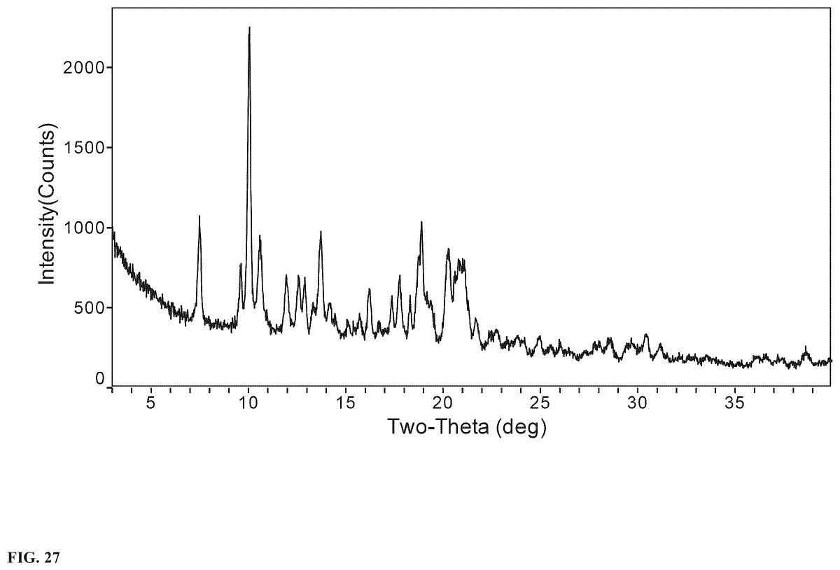

The solid was collected for XRPD analysis. The XRPD pattern of calcium salt Form U of compound (I) is shown in FIG. 27. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 25
X-ray powder diffraction peaks of calcium
salt Form U of compound (I).

| Pos. [°2-theta] | Rel. Int. [%] |
|---|---|
| 7.5 | 36 |
| 9.6 | 15 |
| 10.1 | 100 |
| 10.6 | 27 |
| 11.9 | 17 |
| 12.6 | 18 |
| 12.9 | 17 |
| 13.3 | 8 |
| 13.7 | 34 |
| 14.2 | 9 |
| 15.1 | 5 |
| 15.7 | 7 |
| 16.2 | 15 |
| 16.7 | 4 |
| 17.4 | 13 |
| 17.8 | 20 |
| 18.3 | 12 |
| 18.9 | 39 |
| 19.4 | 12 |
| 20.3 | 27 |
| 21.0 | 26 |
| 21.7 | 7 |
| 22.5 | 5 |
| 22.7 | 5 |
| 23.8 | 4 |
| 24.2 | 5 |
| 25.0 | 6 |
| 25.5 | 3 |
| 26.0 | 5 |
| 26.5 | 3 |
| 27.9 | 4 |
| 28.0 | 6 |
| 28.6 | 6 |
| 29.4 | 4 |
| 29.7 | 5 |
| 30.4 | 7 |
| 31.2 | 5 |
| 32.1 | 3 |
| 32.6 | 3 |
| 33.0 | 3 |
| 36.0 | 3 |
| 36.6 | 4 |
| 37.5 | 2 |
| 38.6 | 4 |

### Example 29: Preparation of Ammonium Salt Form V of Compound (I)

10.34 mg of Form A of compound (I) as prepared in Example 3 was dissolved in 0.3 mL methanol at RT. 2.66 mg of ammonia solution (1.1 eq., 25%-28%) was added to the solution, the mixture was clear but precipitation occurred after 16 hours of agitation. After removing all the solvents, an oil was obtained. 0.05 mL acetonitrile and 0.4 mL IPE was added to the residue, solid formed and was collected by filtration, dried in a vacuum oven for 16 hours.

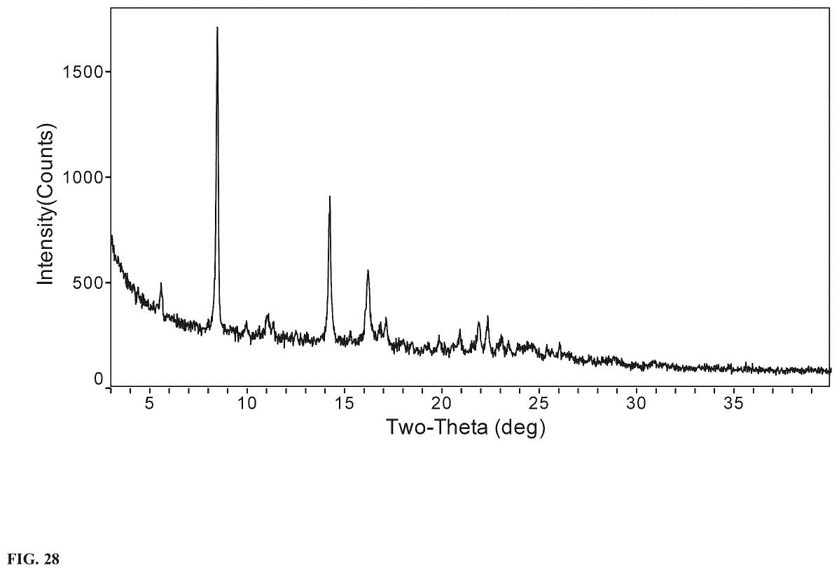

The solid was collected for XRPD analysis. The XRPD pattern of ammonium Salt Form V of compound (I) is shown in FIG. 28. Major peaks and their related intensities in the XRPD pattern are shown in table below.

### Characterization Method

XRPD: Balker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 26
X-ray powder diffraction peaks of ammonium
Salt Form V of compound (I).

| Pos. [°2-theta] | Rel. Int. [%] |
|---|---|
| 5.6 | 10 |
| 8.0 | 4 |
| 8.5 | 100 |
| 10.0 | 5 |
| 10.6 | 4 |
| 11.1 | 8 |
| 11.4 | 5 |
| 14.2 | 48 |
| 15.3 | 4 |
| 16.2 | 23 |
| 16.9 | 7 |
| 17.1 | 8 |
| 19.3 | 3 |
| 19.9 | 6 |
| 20.9 | 8 |
| 21.5 | 3 |
| 21.9 | 10 |
| 22.4 | 11 |
| 23.1 | 6 |
| 23.4 | 4 |
| 24.4 | 4 |
| 24.7 | 3 |
| 25.4 | 4 |
| 26.1 | 5 |
| 28.8 | 3 |
| 35.6 | 3 |

### Example 30: Preparation of Ammonium Salt Form W of Compound (I)

10.47 mg of Form A of compound (I) as prepared in Example 3 was dissolved in 1.0 mL acetonitrile at 50° C., then the solution was cooled down to RT. 2.69 mg of ammonia solution (1.1 eq., 25%-28%) to the solution and the mixture was clear, and precipitation occurred after 16 hours of agitation. The amount of solvent was reduced to 0.2 mL and agitation continued for 3 days at 10° C. The solids were collected by filtration, dried in a vacuum oven for 16 hours.

The solid was collected for XRPD analysis.

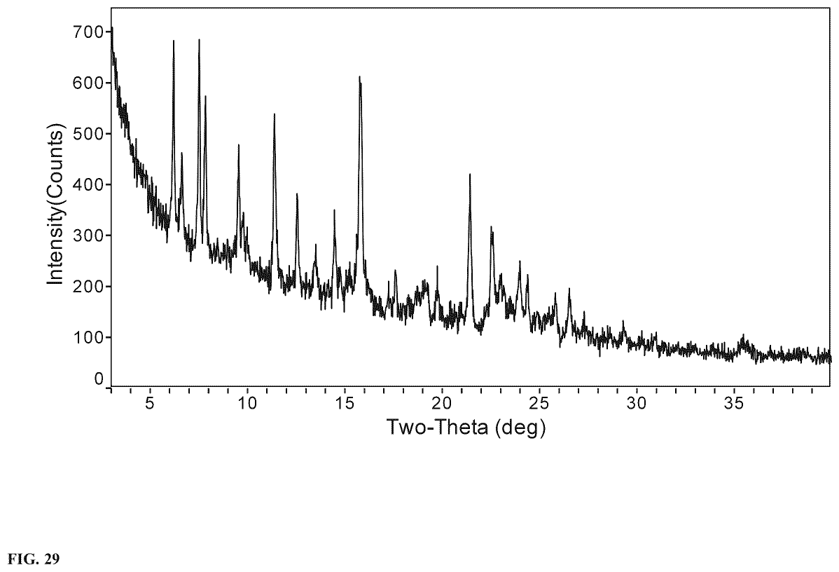

The XRPD pattern of ammonium salt Form W of compound (I) is shown in FIG. 29. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 27
X-ray powder diffraction peaks of ammonium
salt Form W of compound (I).

| Pos. [°2-theta] | Rel. Int. [%] |
|---|---|
| 6.2 | 85 |
| 6.6 | 39 |
| 7.5 | 94 |
| 7.8 | 70 |
| 9.5 | 53 |
| 9.8 | 24 |
| 10.0 | 18 |
| 11.4 | 74 |
| 12.5 | 41 |
| 13.5 | 21 |
| 14.5 | 38 |
| 14.7 | 13 |
| 15.2 | 14 |
| 15.8 | 100 |
| 17.2 | 14 |
| 17.6 | 19 |
| 18.7 | 13 |
| 19.1 | 17 |
| 19.2 | 14 |
| 19.8 | 24 |
| 21.4 | 67 |
| 22.5 | 42 |
| 23.0 | 20 |
| 24.0 | 25 |
| 24.4 | 20 |
| 25.8 | 19 |
| 26.5 | 21 |
| 27.3 | 11 |
| 29.3 | 9 |
| 31.0 | 8 |
| 35.4 | 9 |

### Example 31: Preparation of Ammonium Salt Form X of Compound (I)

400.46 mg of Form A of compound (I) as prepared in Example 3 was dissolved in 30 mL ACN at 65° C. and cooled to RT. 100.35 mg of ammonia solution (1.1 eq., 25%-28%) was added to the solution under agitation, the solution instantly turned cloudy. The suspension was agitated for 17 hours at RT. The solid was collected by filtration, washed with a small amount of acetonitrile and dried in a vacuum oven for 5 hours.

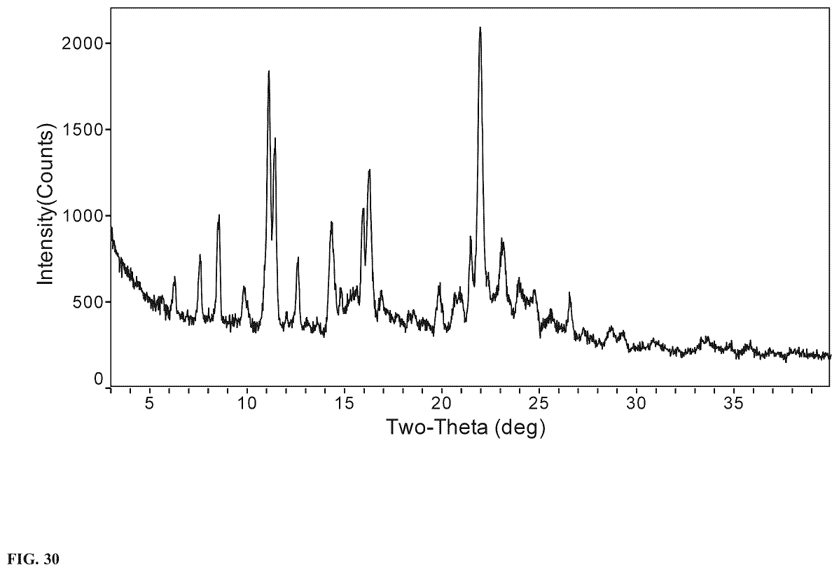

The solid was collected for XRPD analysis. The XRPD pattern of ammonium salt Form X of compound (I) is shown in FIG. 30. Major peaks and their related intensities in the XRPD pattern are shown in table below.

### Characterization Method

XRPD: Bruker D8 Advance diffractometer X-ray powder diffractometer with Cu-Kα radiation. Tube voltage was 40 KV and tube current was 40 mA. Scan range was from 3 to 40 degree 2-theta. The step size was 0.02° at a scanning speed of 6°/min.

TABLE 28
X-ray powder diffraction peaks of ammonium
salt Form X of compound (I).

| Pos. [°2-theta] | Rel. Int. [%] |
|---|---|
| 6.3 | 14 |
| 7.6 | 23 |
| 8.0 | 5 |
| 8.6 | 38 |
| 9.8 | 14 |
| 11.1 | 91 |
| 11.4 | 67 |
| 12.0 | 5 |
| 12.6 | 25 |
| 13.6 | 5 |
| 14.3 | 35 |
| 14.8 | 10 |
| 15.4 | 5 |
| 15.6 | 10 |
| 16.0 | 37 |
| 16.3 | 48 |
| 16.9 | 9 |
| 18.3 | 6 |
| 18.6 | 6 |
| 19.0 | 4 |
| 19.8 | 16 |
| 20.7 | 11 |
| 20.9 | 12 |
| 21.5 | 26 |
| 22.0 | 100 |
| 23.2 | 23 |
| 24.0 | 14 |
| 24.8 | 12 |
| 25.6 | 7 |
| 26.6 | 14 |
| 27.3 | 4 |
| 28.7 | 7 |
| 29.3 | 6 |
| 30.9 | 4 |
| 31.1 | 3 |
| 33.3 | 5 |
| 33.7 | 5 |
| 34.9 | 4 |
| 35.6 | 4 |
| 35.9 | 5 |
| 36.8 | 3 |
| 37.9 | 3 |

### Example 33: Stability of Solid Forms

40 mg of compound (I) in different solid forms were stored in a stability chamber with temperature and humidity controlled at 40 ° C. and 75%-RH, respectively. After 1 month, the samples were analyzed by XRPD to check their solid form and compared with their initial solid form. According to the results shown in Table 29. Form D and sodium salt Form J showed better solid form stability than the original Form D as prepared in Example 1.

TABLE 29
Physical stability data of different solid forms of compound (I)

Physical stability

| Samples | Initial | 40° C./75%-RH, 1 month |
|---|---|---|
| Example 1, Form D of | Form D | solid form change |
| compound (I) | | |
| Example 15, sodium salt | Form J | no solid form change |
| Form J of compound (I) | | |

### Example 34: Apparent Solubility Study

Apparent solubility was determined by suspending 5 mg of compound (I) in different bio-relevant media including pH buffers (50 mM). The suspensions were equilibrated at 25° C. for 24 hours. The suspensions were then filtered through a 0.22 μm PVDF filter into a 2-mL HPLC vial. The quantification of the filtrate was conducted by HPLC with reference to a standard solution. The solubility results of selected novel solid forms in this invention are shown in Table 30. The novel solid forms Form H, Form J, and Form Q of this invention showed higher solubility than Form A at pH7 and pH9.

TABLE 30
Apparent solubility of different solid forms of (I)

| | Sample | | | | | | | |
|---|---|---|---|---|---|---|---|---|
| | | | | | Example 15, | | Example 24, | |
| | Example 3, | | Example 16, | | sodium salt | | potassium salt | |
| | Form A of | | Form H of | | Form J of | | Form Q of | |
| | compound (I) | | compound (I) | | compound (I) | | compound (I) | |
| | Solubility | Final | Solubility | Final | Solubility | Final | Solubility | Final |
| pH | (mg/mL) | pH | (mg/mL) | pH | (mg/mL) | pH | (mg/mL) | pH |
| pH 1 | 0.94 | 1.05 | 1.581 | 1.08 | 1.51 | 1.12 | 1.56 | 1.1 |
| pH 3 | 0.066 | 2.97 | 0.056 | 3.01 | 0.016 | 3.53 | 0.00 | 3.49 |
| pH 5 | 0.004 | 4.99 | 0.006 | 5.03 | 0.0028 | 5.22 | 0.00 | 5.17 |
| pH 7 | 0.072 | 6.98 | 0.244 | 6.97 | 0.69 | 7.34 | 3.58 | 6.97 |
| pH 9 | 4.82 | 8.46 | >10 | 8.90 | >10 | 8.80 | >10 | 8.71 |

### Example 35: Solubility and Stability Study of Form H

Apparent solubility in water was determined by suspending 5 mg of compound (I) in purified water. The suspensions were equilibrated at 25° C. for 24 hours. The suspensions were then filtered through a 0.22 μm PVDF filter into a 2-mL HPLC vial. The quantitation of the filtrate was conducted by HPLC with reference to a standard solution. The solids were analyzed by XRPD. The solubility study results of selected novel solid forms in this invention are shown in Table 31.

TABLE 31
Physical stability data of different solid forms of compound (I)

| | Solubility | Final | XRPD of |
|---|---|---|---|
| Samples | (mg/mL) | pH | residue |
| Example 3, Form A of | 0.05 | 6.97 | Form A |
| compound (I) | | | |
| Example 16, Form H of | 0.453 | 7.35 | Form H |
| compound (I) | | | |

Surprisingly, the monohydrate Form H shows significant higher water solubility than the anhydrate Form A.

20 mg of compound (I) in different solid forms were stored in a stability chamber with temperature and humidity controlled at 25° C. and 60%-RH. After 1 month, the samples were analyzed by XRPD to check their solid form and compared with their initial solid form. Form H showed better stability than the original Form D as prepared in Example 1.

TABLE 32
Physical stability data of different solid forms of compound (I)

Physical stability

| Samples | Initial | 25° C./60%-RH, 1 month |
|---|---|---|
| Example 1, Form D of | Form D | solid form change |
| compound (I) | | |
| Example 16, Form H of | Form H | no solid form change |
| compound (I) | | |

With unexpected higher water solubility and acceptable solid state stability, Form H of compound (I) whose absorption is limited by solubility could be further developed as solid dosage forms to better improve absorption.

1. A solid form of a salt of compound (I), 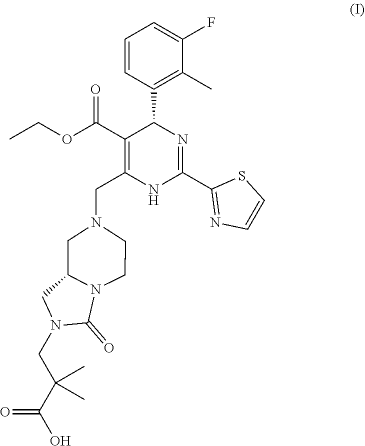 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]
or a solvate thereof.

2. The solid form of claim 1, wherein the solid form is Form J, Form K, Form L, Form M, Form N, Form O, Form P, Form Q, Form R, Form S, Form T, Form U, Form V, Form W or Form X; or a combination thereof.

3. The solid form of claim 2, wherein the solid form is Form J that exhibits an X-ray powder diffraction (XRPD) pattern with characteristic peaks expressed in degrees 2-theta at 7.7°±0.2°, 9.7°±0.2°, 14.7°±0.2°, 15.9°±0.2°, 22.0°±0.2°, 23.4°±0.2°.

4. The solid form of claim 3, wherein the solid form is Form J that exhibits an X-ray powder diffraction (XRPD) pattern with characteristic peaks expressed in degrees 2-theta at 7.7°±0.2°, 9.7°±0.2°, 11.5°±0.2°, 13.0°±0.2°, 14.7°±0.2°, 15.3°±0.2°, 15.9°±0.2°, 16.5°±0.2°, 19.0°±0.2°, 22.0°±0.2°, 22.6°±0.2°, 23.4°±0.2°, 23.9°±0.2°, 24.5°±0.2° and 25.3°±0.2°.

5. The solid form of claim 3, wherein the Form J is the sodium salt of compound (I).

6. The solid form of claim 2, wherein the solid form is Form K that exhibits an X-ray powder diffraction (XRPD) pattern with characteristic peaks expressed in degrees 2-theta at 5.4°±0.2°, 13.3°±0.2°, 15.9°±0.2°, 16.3°±0.2°, 18.0°±0.2° and 22.7°±0.2°.

7. The solid form of claim 7, wherein the solid form is Form K that exhibits an X-ray powder diffraction (XRPD) pattern with characteristic peaks expressed in degrees 2-theta at 5.4°±0.2°, 13.3°±0.2°, 13.8°±0.2°, 14.8°±0.2°, 15.9°±0.2°, 16.3°±0.2°, 18.0°±0.2°, 19.5°±0.2°, 20.0°±0.2°, 21.7°±0.2°, 22.4°±0.2°, 22.7°±0.2°, 23.4°±0.2°, 24.1°±0.2° and 28.0°±0.2°.

8. The solid form of claim 6, wherein the Form K is the hydrochloride salt of compound (I).

9. The solid form of claim 2, wherein the solid form is Form L that exhibits an X-ray powder diffraction (XRPD) pattern with characteristic peaks expressed in degrees 2-theta at 6.0°±0.2°, 11.8°±0.2°, 15.3°±0.2°, 15.8°±0.2°, 18.3°±0.2° and 24.4°±0.2°.

10. The solid form of claim 9, wherein the solid form is Form L that exhibits an X-ray powder diffraction (XRPD) pattern with characteristic peaks expressed in degrees 2-theta at 6.0°±0.2°, 11.2°±0.2°, 11.8°±0.2°, 12.3°±0.2°, 13.1°±0.2°, 15.3°±0.2°, 15.8°±0.2°, 18.3°±0.2°, 18.7°±0.2°, 21.7°±0.2°, 22.5°±0.2°, 23.8°±0.2°, 24.4°±0.2°, 25.7°±0.2° and 27.7°±0.2°.

11. The solid form of claim 9, wherein the Form L is the hydrochloride salt of compound (I).

12. The solid form of claim 2, wherein the solid form is Form M that exhibits an X-ray powder diffraction (XRPD) pattern with characteristic peaks expressed in degrees 2-theta at 5.3°±0.2°, 7.7°±0.2°, 10.7°±0.2°, 17.6°±0.2°, 19.0°±0.2° and 19.2°±0.2°.

13. The solid form of claim 12, wherein the solid form is Form M that exhibits an X-ray powder diffraction (XRPD) pattern with characteristic peaks expressed in degrees 2-theta at 5.3°±0.2°, 7.7°±0.2°, 9.4°±0.2°, 10.7°±0.2°, 15.5°±0.2°, 17.2°±0.2°, 17.6°±0.2°, 19.0°±0.2°, 19.2°±0.2°, 19.8°±0.2° and 24.4°±0.2°.

14. The solid form of claim 12, wherein the Form M is the sulfate salt of compound (I).

15. The solid form of claim 2, wherein the solid form is Form N that exhibits an X-ray powder diffraction (XRPD) pattern with characteristic peaks expressed in degrees 2-theta at 5.3°±0.2°, 10.7°±0.2°, 18.0°±0.2°, 18.7°±0.2°, 19.4°±0.2°, 20.3°±0.2°, 21.5°±0.2° and 24.7°±0.2°.

16. The solid form of claim 15, wherein the Form N is the sulfate salt of compound (I).

17. The solid form of claim 2, wherein the solid form is Form O that exhibits an X-ray powder diffraction (XRPD) pattern with characteristic peaks expressed in degrees 2-theta at 4.9°±0.2°, 10.6°±0.2°, 14.3°±0.2°, 22.4°±0.2° and 22.9°±0.2°.

18. The solid form of claim 17, wherein the solid form is Form O that exhibits an X-ray powder diffraction (XRPD) pattern with characteristic peaks expressed in degrees 2-theta at 4.9°±0.2°, 10.6°±0.2°, 13.2°±0.2°, 14.3°±0.2°, 16.9°±0.2°, 17.9°±0.2°, 19.1°±0.2°, 20.2°±0.2°, 21.1°±0.2°, 22.4°±0.2°, 22.9°±0.2°, 23.9°±0.2° and 24.4°±0.2°.

19. The solid form of claim 17, wherein the Form O is the besylate salt of compound (I).

20. The solid form of claim 2, wherein the solid form is Form P that exhibits an X-ray powder diffraction (XRPD) pattern with characteristic peaks expressed in degrees 2-theta at 3.9°±0.2°, 7.7°±0.2°, 15.3°±0.2°, 21.5°±0.2°, 27.5°±0.2° and 31.8°±0.2°.

21. The solid form of claim 20, wherein the Form P is the potassium salt of compound (I).

22. The solid form of claim 2, wherein the solid form is Form Q that exhibits an X-ray powder diffraction (XRPD) pattern with characteristic peaks expressed in degrees 2-theta at 7.9°±0.2°, 8.7°±0.2°, 13.2°±0.2°, 15.4°±0.2°, 21.8°±0.2°, 26.3°±0.2° and 29.3°±0.2°.

23. The solid form of claim 22, wherein the solid form is Form Q that exhibits an X-ray powder diffraction (XRPD) pattern with characteristic peaks expressed in degrees 2-theta at 7.9°±0.2°, 8.7°±0.2°, 10.5°±0.2°, 11.0°±0.2°, 13.2°±0.2°, 15.4°±0.2°, 16.8°±0.2°, 17.4°±0.2°, 18.1°±0.2°, 18.5°±0.2°, 21.2°±0.2°, 21.8°±0.2°, 26.3°±0.2°, 26.7°±0.2° and 29.3°±0.2°.

24. The solid form of any one of claim 22, wherein the Form Q is the potassium salt of compound (I).

25. The solid form of claim 2, wherein the solid form is Form R that exhibits an X-ray powder diffraction (XRPD) pattern with characteristic peaks expressed in degrees 2-theta at 7.5°±0.2°, 7.8°±0.2°, 9.9°±0.2°, 14.8°±0.2°, 15.4°±0.2°, 15.7°±0.2° and 22.2°±0.2°.

26. The solid form of claim 25, wherein the solid form is Form R that exhibits an X-ray powder diffraction (XRPD) pattern with characteristic peaks expressed in degrees 2-theta at 7.5°±0.2°, 7.8°±0.2°, 8.8°±0.2°, 9.9°±0.2°, 11.2°±0.2°, 11.7°±0.2°, 12.4°±0.2°, 14.8°±0.2°, 15.4°±0.2°, 15.7°±0.2°, 17.2°±0.2°, 22.2°±0.2° and 26.3°±0.2°.

27. The solid form of claim 25, wherein the Form R is the potassium salt of compound (I).

28. The solid form of claim 2, wherein the solid form is Form S that exhibits an X-ray powder diffraction (XRPD) pattern with characteristic peaks expressed in degrees 2-theta at 8.3°±0.2°, 8.7°±0.2°, 13.7°±0.2°, 15.8°±0.2°, 18.0°±0.2° and 21.7°±0.2°.

29. The solid form of claim 28, wherein the solid form is Form S that exhibits an X-ray powder diffraction (XRPD) pattern with characteristic peaks expressed in degrees 2-theta at 8.3°±0.2°, 8.7°±0.2°, 11.0°±0.2°, 11.2°±0.2°, 13.4°±0.2°, 13.7°±0.2°, 15.8°±0.2°, 16.6°±0.2°, 18.0°±0.2°, 20.9°±0.2°, 21.7°±0.2°, 24.5°±0.2°, 26.2°±0.2°, 26.7°±0.2° and 28.6°±0.2°.

30. The solid form of claim 28, wherein the Form S is the potassium salt of compound (I).

31. The solid form of claim 2, wherein the solid form is Form T that exhibits an X-ray powder diffraction (XRPD) pattern with characteristic peaks expressed in degrees 2-theta at 8.0°±0.2°, 10.8°±0.2°, 11.1°±0.2°, 13.3°±0.2°, 15.5°±0.2°, 21.5°±0.2° and 31.6°±0.2°.

32. The solid form of claim 31, wherein the Form T is the calcium salt of compound (I).

33. The solid form of claim 2, wherein the solid form is Form U that exhibits an X-ray powder diffraction (XRPD) pattern with characteristic peaks expressed in degrees 2-theta at 7.5°±0.2°, 10.1°±0.2°, 10.6°±0.2°, 13.7°±0.2°, 18.9°±0.2°, 20.3°±0.2° and 21.0°±0.2°.

34. The solid form of claim 33, wherein the solid form is Form U that exhibits an X-ray powder diffraction (XRPD) pattern with characteristic peaks expressed in degrees 2-theta at 7.5°±0.2°, 9.6°±0.2°, 10.1°±0.2°, 10.6°±0.2°, 11.9°±0.2°, 12.6°±0.2°, 12.9°±0.2°, 13.7°±0.2°, 16.2°±0.2°, 17.8°±0.2°, 18.9°±0.2°, 20.3°±0.2° and 21.0°±0.2° .

35. The solid form of claim 33, wherein the Form U is the calcium salt of compound (I).

36. The solid form of claim 2, wherein the solid form is Form V that exhibits an X-ray powder diffraction (XRPD) pattern with characteristic peaks expressed in degrees 2-theta at 5.6°±0.2°, 8.5°±0.2°, 14.2°±0.2°, 16.2°±0.2°, 21.9°±0.2° and 22.4°±0.2°.

37. The solid form of claim 36, wherein the Form V is the ammonium salt of compound (I).

38. The solid form of claim 2, wherein the solid form is Form W that exhibits an X-ray powder diffraction (XRPD) pattern with characteristic peaks expressed in degrees 2-theta at 6.2°±0.2°, 7.5°±0.2°, 7.8°±0.2°, 11.4°±0.2°, 15.8°±0.2° and 21.4°±0.2°.

39. The solid form of claim 38, wherein the solid form is Form W that exhibits an X-ray powder diffraction (XRPD) pattern with characteristic peaks expressed in degrees 2-theta at 6.2°±0.2°, 6.6°±0.2°, 7.5°±0.2°, 7.8°±0.2°, 9.5°±0.2°, 9.8°±0.2°, 11.4°±0.2°, 12.5°±0.2°, 13.5°±0.2°, 14.5°±0.2°, 15.8°±0.2°, 19.8°±0.2°, 21.4°±0.2°, 22.5°±0.2°, 24.0°±0.2° and 26.5°±0.2°.

40. The solid form of claim 38, wherein the Form W is the ammonium salt of compound (I).

41. The solid form of claim 2, wherein the solid form is Form X that exhibits an X-ray powder diffraction (XRPD) pattern with characteristic peaks expressed in degrees 2-theta at 8.6°±0.2°, 11.1°±0.2°, 11.4°±0.2°, 14.3°±0.2°, 16.0°±0.2°, 16.3°±0.2° and 22.0°±0.2°.

42. The solid form of claim 41, wherein the solid form is Form X that exhibits an X-ray powder diffraction (XRPD) pattern with characteristic peaks expressed in degrees 2-theta at 7.6°±0.2°, 8.6°±0.2°, 11.1°±0.2°, 11.4°±0.2°, 12.6°±0.2°, 14.3°±0.2°, 16.0°±0.2°, 16.3°±0.2°, 19.8°±0.2°, 21.5°±0.2°, 22.0°±0.2° and 23.2°±0.2°.

43. The solid form of claim 41, wherein the Form X is the ammonium salt of compound (I).

44. A pharmaceutical composition comprising the solid form of claim 2 and a pharmaceutically acceptable carrier, excipient, diluent, adjuvant, vehicle or a combination thereof.

45. A method for the treatment or prophylaxis of HBV infection or a disease caused by HBV infection, which method comprises administering a therapeutically effective amount of the solid form as defined in claim 2.
